SEQUENCE LISTING (24 sequences)

SEQ ID NO: 1 - protein, 444 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 443 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 3 - protein, 221 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 5 - protein, 117 aa, Artificial Sequence
  Synthetic Construct
QVQLVQSGAEVKKPGASVKVSCKASGYTFTSYWINWVRQAPGQGLEWMGNIWPGSSLTNYNEKFKNRVTMTRDTSTSTVYMELSSLRSEDTAVYYCARLLTGTFAYWGQGTLVTVSS

SEQ ID NO: 6 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 7 - protein, 432 aa, Homo sapiens
QVQLVESGGGWQPGRSLRLDCKASGITFSNSGMHWVRQAPGKGLEWVAVRWYDGSKRYYADSVKGRFTISRDNSKNTLFLQMNSLRAEDTAVYYCATNDDYWGQGTLVTVSSASTKGPSVFPLAPCSRSTSESTAALGCLVDYFPEPVTVSWNSGALTSGVHTFPAVLQSSGLYSLSSVVTVPSSSLGTTYTCNVDHKPSNTKVDRVESYGPPCPPCPAPEFLGGPSVFLFPPKPKDTLMISRTPEVTCWVDVSQEDPEVQFNWYYDGVEVHNATKPREEQFNSTYRVVSVLTVLHQDWLNGKEYKCKVSNKGLPSSIEKTISKAGQPREPQVYTLPPSQEEMTKNQVSLTCLVKGFYPSDIAVEWESNGQPEKNYKTTPPVLDSDGSFFLYSRLTVDKSRWQEGNVFSCSVMHEALHNHYTQKSLSLSLGK

SEQ ID NO: 8 - protein, 208 aa, Homo sapiens
EIVLTQSPATLSLSPGERATLSCRASQSVSSYLAWYQQPGQAPRLLIYDASNRATGIPARFSGSGSGTDFTLTISSLEPEDFAVYYCQQSSNWPRTFGQGTKVEIRTVAAPSVFIFPPSDEQLSGTASVVCLLNNFYPREAVQWKVDNALQSGNSQESVTEQDSDSTYSLSSTLTLSKADYEKHKVYACEVTHQGLSSPVTSFNRGEC

SEQ ID NO: 9 - protein, 447 aa, Artificial Sequence
  Synthetic Construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 218 aa, Artificial Sequence
  Synthetic Construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 432 aa, Artificial Sequence
  Synthetic Construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 118 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 13 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 117 aa, Artificial Sequence
  Synthetic Construct
EVQLLESGGGLVQPGGSLRLSCAASGFTFSSYIMWVRQAPGKGLEWVSSIYPSGGITFYADKGRFTISRDNSKNTLYLQMNSLRAEDTAVYYCARIKLGTVTTVDYWGQGTLVTVSS

SEQ ID NO: 15 - protein, 110 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 16 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 17 - protein, 5 aa, Artificial Sequence
  Synthetic Construct
SYIMM

SEQ ID NO: 18 - protein, 11 aa, Artificial Sequence
  Synthetic Construct
SIYPSGGITFY

SEQ ID NO: 19 - protein, 11 aa, Artificial Sequence
  Synthetic Construct
IKLGTVTTVDY

SEQ ID NO: 20 - protein, 14 aa, Artificial Sequence
  Synthetic Construct
TGTSSDVGGYNYVS

SEQ ID NO: 21 - protein, 7 aa, Artificial Sequence
  Synthetic Construct
DVSNRPS

SEQ ID NO: 22 - protein, 10 aa, Artificial Sequence
  Synthetic Construct
SSYTSSSTRV

SEQ ID NO: 23 - protein, 450 aa, Artificial Sequence
  Synthetic Construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 216 aa, Artificial Sequence
  Synthetic Construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2021/0077463 A1
(43) Pub. Date: Mar. 18, 2021

(54) Methods and Combination Therapy to Treat Cancer

(71) Applicants: PFIZER INC., New York, NY (US); MERCK PATENT GMBH, Darmstadt (DE); ARRAY BIOPHARMA INC., Boulder, CO (US)

(72) Inventors: Christoffel Hendrik Boshoff, New York, NY (US); Rossano Cesari, Latina (IT); Cristian Massacesi, Summit, NJ (US); Nuzhat Pathan, San Diego, CA (US); Patrice A. Lee, Boulder, CO (US); Shannon L. Winski, Boulder, CO (US)

(21) Appl. No.: 16/772,306

(22) PCT Filed: Dec. 17, 2018
(86) PCT No.: PCT/IB2018/060181

§ 371 Date: Jun. 12, 2020

Related U.S. Application Data
(60) Provisional application No. 62/607,190, filed on Dec. 18, 2017.

(51) Int. Cl. A61K 31/4184; A61K 39/395; A61K 31/5025; A61P 35/00; A61K 9/00

(52) U.S. Cl. CPC A61K 31/4184; A61K 39/3955; A61K 2039/545; A61P 35/00; A61K 9/0053; A61K 31/5025

(57) ABSTRACT

This invention relates to a method of treating cancer by administering a combination therapy comprising a combination of a MEK inhibitor and a PD-1 axis binding antagonist, or a combination of a MEK inhibitor and a PARP inhibitor, or a combination of a MEK inhibitor and a PD-1 axis binding antagonist and a PARP inhibitor to a patient in need thereof.

### FIELD

The present invention relates to methods and combination therapies useful for the treatment of cancer. In particular, this invention relates to methods and combination therapies for treating cancer by administering a combination therapy comprising a combination of a MEK inhibitor and a PD-1 axis binding antagonist, or a combination of a MEK inhibitor and a PARP inhibitor, or a combination of a MEK inhibitor and a PD-1 axis binding antagonist and a PARP inhibitor. Pharmaceutical uses of the combination of the present invention are also described.

### BACKGROUND

PD-L1 is overexpressed in many cancers and is often associated with poor prognosis (Okazaki T et al., Intern. Immun. 2007 19(7):813) (Thompson R H et al., Cancer Res 2006, 66(7):3381). Interestingly, the majority of tumor infiltrating T lymphocytes predominantly express PD-1, in contrast to T lymphocytes in normal tissues and peripheral blood. PD-1 on tumor-reactive T cells can contribute to impaired antitumor immune responses (Ahmadzadeh et al., Blood 2009 1 14(8): 1537). This may be due to exploitation of PD-L1 signaling mediated by PD-L1 expressing tumor cells interacting with PD-1 expressing T cells to result in attenuation of T cell activation and evasion of immune surveillance (Sharpe et al., Nat Rev 2002, Keir M E et al., 2008 Annu. Rev. Immunol. 26:677). Therefore, inhibition of the PD-L1/PD-1 interaction may enhance CD8+ T cell-mediated killing of tumors.

The inhibition of PD-1 axis signaling through its direct ligands (e.g., PD-L1, PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. Other advantageous therapeutic treatment regimens could combine blockade of PD-1 receptor/ligand interaction with other anti-cancer agents. There remains a need for such an advantageous therapy for treating, stabilizing, preventing, and/or delaying development of various cancers.

Several PD-1 axis antagonists, including the PD-1 antibodies nivolumab (Opdivo), pembrolizumab (Keytruda) and PD-L1 antibodies avelumab (Bavencio), durvalumab (Imfinzi), and azezolizumab (Tecentriq) were approved by the U.S. Food and Drug Administration (FDA) for the treatment of cancer in recent years.

Mitogen-activated protein kinase kinase (also known as MAP2K, MEK or MAPKK) is a kinase enzyme which phosphorylates mitogen-activated protein kinase (MAPK). The MAPK signaling pathways play critical roles in cell proliferation, survival, differentiation, motility and angiogenesis. Four distinct MAPK signaling cascades have been identified, one of which involves extracellular signal-regulated kinases ERK1 and ERK2 and their upstream molecules MEK1 and MEK2. (Akinleye, et al., Journal of Hematology & Oncology 2013 6:27). Inhibitors of MEK1 and MEK2 have been the focus of antitumor drug discoveries, with trametinib being approved by the FDA to treat BRAF mutant melanoma and many other MEK1/2 inhibitors being studied in clinical studies.

Poly (ADP-ribose) polymerase (PARP) engages in the naturally occurring process of DNA repair in a cell. PARP inhibition has been shown to be an effective therapeutic strategy against tumors associated with germline mutation in double-strand DNA repair genes by inducing synthetic lethality (Sonnenblick, A., et al., Nat Rev Clin Oncol, 2015. 12(1), 27-4). One PARP inhibitor (PARPi), olaparib, was approved by the FDA in 2014 for the treatment of germline BRCA-mutated (gBRCAm) advanced ovarian cancer. More recently, the PARP inhibitors niraparib and rucaparib were also approved by the FDA for treatment of ovarian cancer

There remains a need of finding advantageous combination therapies for treating cancer patients, or a particular population of cancer patients, and potentially with particularized dosing regimens, to improve clinical anti-tumor activity as compared to single agent treatment or double agent treatment, and to optionally improve the combination safety profile.

### SUMMARY

Each of the embodiments described below can be combined with any other embodiment described herein not inconsistent with the embodiment with which it is combined. Furthermore, each of the embodiments described herein envisions within its scope pharmaceutically acceptable salts of the compounds described herein. Accordingly, the phrase “or a pharmaceutically acceptable salt thereof” is implicit in the description of all compounds described herein. Embodiments within an aspect as described below can be combined with any other embodiments not inconsistent within the same aspect or a different aspect.

In one embodiment, provided herein is a combination therapy comprising therapeutically effective amounts, independently, of a MEK inhibitor, and a PD-1 axis binding antagonist.

In one embodiment, provided herein is a combination therapy comprising therapeutically effective amounts, independently, of a MEK inhibitor, a PD-1 axis binding antagonist, and a PARP inhibitor.

In one embodiment, the invention provides a method for treating cancer comprising administering to a patient in need thereof an amount of a PARP inhibitor, an amount of a PD-1 axis binding antagonist, and an amount of a MEK inhibitor, wherein the amounts together are effective in treating cancer.

In one aspect of this embodiment and in combination with any other aspects not inconsistent, the cancer of the patient is a RAS mutant cancer. In some embodiments, the cancer is KRAS mutant cancer or KRAS associated cancer. In some embodiments, the cancer is HRAS mutant cancer or HRAS associated cancer. In some embodiments, the cancer is NRAS mutant cancer or NRAS associated cancer.

In another aspect of this embodiment and in combination with any other aspects not inconsistent, the PD-1 axis antagonist is an anti PD-1 antibody selected from nivolumab and pembrolizumab. In some embodiments, the PD-1 axis antagonist is an anti PD-L1 antibody selected from avelumab, durvalumab and atezolizumab. In some embodiment, the PD-1 axis binding antagonist is avelumab.

In another aspect of this embodiment and in combination with any other aspects not inconsistent, the PARP inhibitor is selected from the group consisting of olaparib, niraparib, BGB-290 and talazoparib, or a pharmaceutically acceptable salt thereof. In some embodiments, the PARP inhibitor is talazoparib, or a pharmaceutically acceptable salt thereof. In some embodiments, the PARP inhibitor is talazoparib tosylate.

In another aspect of this embodiment and in combination with any other aspects not inconsistent the MEK inhibitor is selected from the group consisting of trametinib, cobimetinib, refametinib, selumetinib, binimetinib, PD0325901, PD184352, PD098059, U0126, CH4987655, CH5126755 and GDC623, or pharmaceutically acceptable salts thereof. In some embodiments, the MEK inhibitor is binimetinib or a pharmaceutically acceptable salt thereof.

In another aspect of this embodiment and in combination with any other aspects not inconsistent, the cancer is pancreatic cancer. In some embodiments, the cancer is metastatic pancreatic cancer, wherein the patient has received at least one prior line of chemotherapy for the cancer. In some embodiments, the chemotherapy is FOLFIRINOX (a combination of folinic acid, 5-fluorouracil, irinotecan, and oxaliplatin), gemcitabine, or gemcitabine in combination with nab-paclitaxel.

In another aspect of this embodiment and in combination with any other aspects not inconsistent, the cancer is non-small cell lung cancer (NSCLC). In some embodiments, the cancer is locally advanced or metastatic NSCLC. In some embodiments, the patient has received at least 1 prior line of treatment for the locally advanced or metastatic NSCLC. In some embodiments, the NSCLC is KRAS mutant cancer or KRAS associated cancer. In some embodiments, the NSCLC cancer is KRAS mutant cancer. In some embodiments, the cancer is locally advanced or metastatic NSCLC, wherein the patient has received at least 1 prior line of treatment for the locally advanced or metastatic NSCLC, and wherein the NSCLC is KRAS mutant cancer. In some embodiments, the prior treatment is platinum-based chemotherapy, docetaxel, a PD-1 axis antagonist or a combination of chemotherapy with a PD-1 axis antagonist.

In another aspect of this embodiment and in combination with any other aspects not inconsistent, the cancer is KRAS mutant cancer including but not limited to colorectal cancer and gastric cancer.

In another embodiment, the invention provides a method for treating cancer comprising administering to a patient in need thereof an amount of a PARP inhibitor, an amount of a PD-1 axis binding antagonist, and an amount of a MEK inhibitor, wherein the PARP inhibitor is talazoparib or a pharmaceutically acceptable salt thereof, the PD-1 axis antagonist is avelumab, and the MEK inhibitor is binimetinib or a pharmaceutically acceptable salt thereof, wherein the amounts together are effective in treating cancer.

In one aspect of this embodiment and in combination with any other aspects not inconsistent, the PARP inhibitor is talazoparib tosylate, and the MEK inhibitor is binimetinib or a pharmaceutically acceptable salt thereof. In one embodiment, the MEK inhibitor is binimetinib as the free base. In one embodiment, the MEK inhibitor is a pharmaceutically acceptable salt of binimetinib.

In one aspect of this embodiment and in combination of any other aspect not inconsistent, talazoparib or a pharmaceutically acceptable salt thereof is administered orally in the amount of about 0.5 mg QD, about 0.75 mg QD or about 1.0 mg QD.

In another aspect of this embodiment, and in combination of any other aspect not inconsistent, avelumab is administered intravenously in the amount of about 800 mg every 2 weeks (Q2W) or about 10 mg/kg every 2 weeks (Q2W). In one embodiment, avelumab is administered intravenously over 60 minutes.

In another aspect of this embodiment, and in combination of any other aspect not inconsistent, the MEK inhibitor is binimetinib as the free base. In one embodiment, the MEK inhibitor is crystallized binimetinib, that is the crystallized form of the free base of binimetinib. In one embodiment, binimetinib is orally administered daily in the amount of (a) about 30 mg BID or about 45 mg twice a day (BID), or (b) orally administered daily in the amount of about 30 mg BID or about 45 mg BID for three weeks followed by one week without administration of binimetinib in at least one treatment cycle of 28 days.

In one aspect of this embodiment and in combination with any other aspects not inconsistent, the cancer of the patient is a RAS mutant cancer. In some embodiments, the cancer is KRAS mutant cancer or KRAS associated cancer. In some embodiments, the cancer is HRAS mutant cancer or HRAS associated cancer. In some embodiments, the cancer is NRAS mutant cancer or NRAS associated cancer.

In another aspect of this embodiment and in combination with any other aspects not inconsistent, the cancer is pancreatic cancer. In some embodiments, the cancer is metastatic pancreatic cancer, wherein the patient has received at least one prior line of chemotherapy for the cancer. In some embodiments, the chemotherapy is FOLFIRINOX (a combination of folinic acid, 5-fluorouracil, irinotecan, and oxaliplatin), gemcitabine, or gemcitabine in combination with nab-paclitaxel.

In another aspect of this embodiment and in combination with any other aspects not inconsistent, the cancer is non-small cell lung cancer (NSCLC). In some embodiments, the cancer is locally advanced or metastatic NSCLC. In some embodiments, the patient has received at least 1 prior line of treatment for the locally advanced or metastatic NSCLC. In some embodiments, the NSCLC is KRAS mutant cancer or KRAS associated cancer. In some embodiments, the NSCLC cancer is KRAS mutant cancer. In some embodiments, the cancer is locally advanced or metastatic NSCLC, wherein the patient has received at least 1 prior line of treatment for the locally advanced or metastatic NSCLC, and wherein the NSCLC is KRAS mutant cancer. In some embodiments, the prior treatment is platinum-based chemotherapy, docetaxel, a PD-1 axis antagonist or a combination of chemotherapy with a PD-1 axis antagonist.

In another aspect of this embodiment and in combination with any other aspects not inconsistent, the cancer is KRAS mutant cancer including but not limited to colorectal cancer and gastric cancer.

In another embodiment, the invention provides a method for treating cancer comprising administering to a patient in need thereof an amount of a PARP inhibitor, an amount of a PD-1 axis binding antagonist, and an amount of a MEK inhibitor, wherein the PARP inhibitor is talazoparib or a pharmaceutically acceptable salt thereof and is administered orally in the amount of about 0.5 mg QD, about 0.75 mg QD or about 1.0 mg QD, the PD-1 axis antagonist is avelumab and is administered intravenously in the amount of about 800 mg Q2W or about 10 mg/kg Q2W, the MEK inhibitor is binimetinib or a pharmaceutically acceptable salt thereof and is administered orally in the amount of (a) about 30 mg BID or about 45 mg BID, or (b) about 30 mg BID or about 45 mg BID for three weeks followed by one week without administration of binimetinib in at least one treatment cycle of 28 days.

In one aspect of this embodiment and in combination with any other aspects not inconsistent, the PARP inhibitor is talazoparib tosylate, the MEK inhibitor is binimetinib, and the PD-1 axis binding antagonist is avelumab.

In one aspect of this embodiment and in combination with any other aspects not inconsistent, the cancer of the patient is a RAS mutant cancer. In some embodiments, the cancer is KRAS mutant cancer or KRAS associated cancer. In some embodiments, the cancer is HRAS mutant cancer or HRAS associated cancer. In some embodiments, the cancer is NRAS mutant cancer or NRAS associated cancer.

In another aspect of this embodiment and in combination with any other aspects not inconsistent, the cancer is pancreatic cancer. In some embodiments, the cancer is metastatic pancreatic cancer, wherein the patient has received at least one prior line of chemotherapy for the cancer. In some embodiments, the chemotherapy is FOLFIRINOX (a combination of folinic acid, 5-fluorouracil, irinotecan, and oxaliplatin), gemcitabine, or gemcitabine in combination with nab-paclitaxel.

In another aspect of this embodiment and in combination with any other aspects not inconsistent, the cancer is non-small cell lung cancer (NSCLC). In some embodiments, the cancer is locally advanced or metastatic NSCLC. In some embodiments, the patient has received at least 1 prior line of treatment for the locally advanced or metastatic NSCLC. In some embodiments, the NSCLC is KRAS mutant cancer or KRAS associated cancer. In some embodiments, the NSCLC cancer is KRAS mutant cancer. In some embodiments, the cancer is locally advanced or metastatic NSCLC, wherein the patient has received at least 1 prior line of treatment for the locally advanced or metastatic NSCLC, and wherein the NSCLC is KRAS mutant cancer. In some embodiments, the prior treatment is platinum-based chemotherapy, docetaxel, a PD-1 axis antagonist or a combination of chemotherapy with a PD-1 axis antagonist.

In another aspect of this embodiment and in combination with any other aspects not inconsistent, the cancer is KRAS mutant cancer including but not limited to colorectal cancer and gastric cancer.

In one embodiment, the invention provides a method for treating cancer comprises administering to a patient in need thereof a combination therapy comprising therapeutically effective amounts, independently, of a MEK inhibitor, which is binimetinib, a PD-L1 binding antagonist which is avelumab, and a PARP inhibitor which is talazoparib or a pharmaceutically salt thereof.

In one embodiment, provided herein is a method for treating cancer comprising administering to a patient in need thereof a combination therapy comprising therapeutically effective amounts, independently, of a MEK inhibitor, which is binimetinib, wherein binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days; a PD-1 axis binding antagonist which is avelumab, wherein avelumab is administered intravenously over 60 minutes in the amount of about 800 mg every Q2W or about 10 mg/kg Q2W; and a PARP inhibitor, which is talozaparib or pharmaceutically acceptable salt thereof, and is administered orally in the amount of about 0.5 mg QD, about 0.75 mg QD or about 1.0 mg QD, In one embodiment, the PARP inhibitor is talazoparib tosylate.

In another embodiment, the invention provides a method for treating cancer comprising administering to a patient in need thereof an amount of a PD-1 axis binding antagonist, and an amount of a MEK inhibitor, wherein the PD-1 axis antagonist is avelumab, the MEK inhibitor is binimetinib or a pharmaceutically acceptable salt thereof, wherein the amounts together are effective in treating cancer.

In one aspect of this embodiment and in combination with any other aspects not inconsistent, avelumab is administered intravenously in the amount of about 800 mg Q2W or about 10 mg/kg Q2W, binimetinib or a pharmaceutically acceptable salt thereof is administered orally in the amount of (a) about 30 mg BID or about 45 mg BID, or (b) about 30 mg BID or about 45 mg BID for three weeks followed by one week without administration of binimetinib in at least one treatment cycle of 28 days.

In one aspect of this embodiment and in combination with any other aspects not inconsistent, the cancer of the patient is a RAS mutant cancer. In some embodiments, the cancer is KRAS mutant cancer or KRAS associated cancer. In some embodiments, the cancer is HRAS mutant cancer or HRAS associated cancer. In some embodiments, the cancer is NRAS mutant cancer or NRAS associated cancer.

In another aspect of this embodiment and in combination with any other aspects not inconsistent, the cancer is pancreatic cancer. In some embodiments, the cancer is metastatic pancreatic cancer, wherein the patient has received at least one prior line of chemotherapy for the cancer. In some embodiments, the chemotherapy is FOLFIRINOX (a combination of folinic acid, 5-fluorouracil, irinotecan, and oxaliplatin), gemcitabine, or gemcitabine in combination with nab-paclitaxel.

In another aspect of this embodiment and in combination with any other aspects not inconsistent, the cancer is non-small cell lung cancer (NSCLC). In some embodiments, the cancer is locally advanced or metastatic NSCLC. In some embodiments, the patient has received at least 1 prior line of treatment for the locally advanced or metastatic NSCLC. In some embodiments, the NSCLC is KRAS mutant cancer or KRAS associated cancer. In some embodiments, the NSCLC cancer is KRAS mutant cancer. In some embodiments, the cancer is locally advanced or metastatic NSCLC, wherein the patient has received at least 1 prior line of treatment for the locally advanced or metastatic NSCLC, and wherein the NSCLC is KRAS mutant cancer. In some embodiments, the prior treatment is platinum-based chemotherapy, docetaxel, a PD-1 axis antagonist or a combination of chemotherapy with a PD-1 axis antagonist.

In another aspect of this embodiment and in combination with any other aspects not inconsistent, the cancer is KRAS mutant cancer including but not limited to colorectal cancer and gastric cancer.

In another embodiment, the invention provides a method for treating cancer comprising administering to a patient in need thereof an amount of a PARP inhibitor, and an amount of a MEK inhibitor, wherein the PARP inhibitor is talazoparib or a pharmaceutically acceptable salt thereof, the MEK inhibitor is binimetinib or a pharmaceutically acceptable salt thereof, wherein the amounts together are effective in treating cancer.

In one aspect of this embodiment and in combination with any other aspects not inconsistent, talazoparib or a pharmaceutically acceptable salt thereof is administered orally in the amount of about 0.5 mg QD, about 0.75 mg QD or about 1.0 mg QD, binimetinib or a pharmaceutically acceptable salt is administered orally in the amount of (a) about 30 mg BID or about 45 mg BID, or (b) about 30 mg BID or about 45 mg BID for three weeks followed by one week without administration of binimetinib in at least one treatment cycle of 28 days.

In one aspect of this embodiment and in combination with any other aspects not inconsistent, the cancer of the patient is a RAS mutant cancer. In some embodiments, the cancer is KRAS mutant cancer or KRAS associated cancer. In some embodiments, the cancer is HRAS mutant cancer or HRAS associated cancer. In some embodiments, the cancer is NRAS mutant cancer or NRAS associated cancer.

In another aspect of this embodiment and in combination with any other aspects not inconsistent, the cancer is pancreatic cancer. In some embodiments, the cancer is metastatic pancreatic cancer, wherein the patient has received at least one prior line of chemotherapy for the cancer. In some embodiments, the chemotherapy is FOLFIRINOX (a combination of folinic acid, 5-fluorouracil, irinotecan, and oxaliplatin), gemcitabine, or gemcitabine in combination with nab-paclitaxel.

In another aspect of this embodiment and in combination with any other aspects not inconsistent, the cancer is non-small cell lung cancer (NSCLC). In some embodiments, the cancer is locally advanced or metastatic NSCLC. In some embodiments, the patient has received at least 1 prior line of treatment for the locally advanced or metastatic NSCLC. In some embodiments, the NSCLC is KRAS mutant cancer or KRAS associated cancer. In some embodiments, the NSCLC cancer is KRAS mutant cancer. In some embodiments, the cancer is locally advanced or metastatic NSCLC, wherein the patient has received at least 1 prior line of treatment for the locally advanced or metastatic NSCLC, and wherein the NSCLC is KRAS mutant cancer. In some embodiments, the prior treatment is platinum-based chemotherapy, docetaxel, a PD-1 axis antagonist or a combination of chemotherapy with a PD-1 axis antagonist.

In another aspect of this embodiment and in combination with any other aspects not inconsistent, the cancer is KRAS mutant cancer including but not limited to colorectal cancer and gastric cancer.

In another aspect of all the foregoing embodiments of this invention, and in combination with any other aspects not inconsistent, the cancer has a tumor proportion score for PD-L1 expression of less than about 1%, or equal or over about 1%, 5%, 10%, 25%, 50%, 75% or 80%.

In another aspect of all the foregoing embodiments of this invention, and in combination with any other aspects not inconsistent, the cancer has a loss of heterozygosity (LOH) score of about 5% or more, 10% or more, 14% or more 15% or more, 20% or more, or 25% or more.

In another aspect of this embodiment and in combination with any other aspects not inconsistent, the cancer is DDR defect positive in at least one DDR gene. In some embodiments, the cancer is DDR defect positive in at least one DDR gene selected from BRCA1, BRCA2, ATM, ATR, CHK2, PALB2, MRE11A, NMB RAD51C, MLH1, FANCA and FANC.

In another aspect of all the foregoing embodiments of this invention, and in combination with any other aspects not inconsistent, the cancer has a HRD score of about 20 or above, 25 or above, 30 or above, 35 or above, 40 or above, 42 or above, 45 or above, or 50 or above.

In another aspect of all the foregoing embodiments of this invention, and in combination with any other aspects not inconsistent, the method provides an objective response rate of the patients under the treatment of at least about 20%, at least about 30%, at least about 40%, at least about 50%.

In another aspect of all the foregoing embodiments of this invention, and in combination with any other aspects not inconsistent, the method provides a median overall survival time of the patients under the treatment of at least about 1 month, at least about 2 months, at least about 3 months, at least about 4 months, at least about 5 months, at least about 6 months, at least about 7 months, at least about 8 months, at least about 9 months, at least about 10 months or at least about 11 months.

### DETAILED DESCRIPTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

### General Techniques and Definitions

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R.I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., 1RL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

“About” when used to modify a numerically defined parameter (e.g., the dose of a MEK inhibitor, a PD-1 axis binding antagonist, or a PARP inhibitor, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg. “About” when used at the beginning of a listing of parameters is meant to modify each parameter. For example, about 0.5 mg, 0.75 mg or 1.0 mg means about 0.5 mg, about 0.75 mg or about 1.0 mg. Likewise, about 5% or more, 10% or more, 15% or more, 20% or more, and 25% or more means about 5% or more, about 10% or more, about 15% or more, about 20% or more, and about 25% or more.

“Administration”, “administering”, “treating”, and “treatment,” as it applies to a patient, individual, animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, and rabbit) and most preferably a human. “Treatment” and “treating”, as used in a clinical setting, is intended for obtaining beneficial or desired clinical results. For purposes of this invention, beneficial or desired clinical results include, but are not limited to, one or more of the following: reducing the proliferation of (or destroying) neoplastic or cancerous cells, inhibiting metastasis of neoplastic cells, shrinking or decreasing the size of a tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolonging survival of patients having a disease (e.g., cancer). For example, treatment can be the diminishment of one or several symptoms of a disorder or complete eradication of a disorder, such as cancer. Within the meaning of the present invention, the term “treat” also denotes to arrest, delay the onset (i.e., the period prior to clinical manifestation of a disease) and/or reduce the risk of developing or worsening a disease. “Treatment” can also mean prolonging survival as compared to expected survival if not receiving treatment, for example, an increase in overall survival (OS) compared to a subject not receiving treatment as described herein, and/or an increase in progression-free survival (PFS) compared to a subject not receiving treatment as described herein. The term “treating” can also mean an improvement in the condition of a subject having a cancer, e.g., one or more of a decrease in the size of one or more tumor(s) in a subject, a decrease or no substantial change in the growth rate of one or more tumor(s) in a subject, a decrease in metastasis in a subject, and an increase in the period of remission for a subject (e.g., as compared to the one or more metric(s) in a subject having a similar cancer receiving no treatment or a different treatment, or as compared to the one or more metric(s) in the same subject prior to treatment). Additional metrics for assessing response to a treatment in a subject having a cancer are disclosed herein below.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen binding fragments thereof (such as Fab, Fab′, F (ab′) 2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen (e.g., PD-L1). Antigen binding functions of an antibody can be performed by fragments of an intact antibody. Examples of binding fragments encompassed within the term “antigen binding fragment” of an antibody include Fab; Fab′; F (ab′) 2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “preferentially binds” or “specifically binds” (used interchangeably herein) to a target (e.g., PD-L1 protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PD-L1 epitope is an antibody that binds this epitope with greater affinity, avidity, more readily, and/or with greater duration than it binds to other PD-L1 epitopes or non-PD-L1 epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Iazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The term “PD-1 axis binding antagonist” as used herein refers to a molecule that inhibits the interaction of a PD-1 axis binding partner with one or more of its binding partners, so as to remove T-cell dysfunction resulting from signaling on the PD-1 signaling axis, with a result being to restore or enhance T-cell function. As used herein, a PD-1 axis binding antagonist includes a PD-1 binding antagonist, a PD-L1 binding antagonist and a PD-L2 binding antagonist. In one embodiment, the PD-1 axis binding antagonist is a PD-L1 binding antagonist. In one embodiment, the PD-L1 binding antagonist is avelumab.

Table 2 below provides a list of the amino acid sequences of exemplary PD-1 axis binding antagonists for use in the treatment method, medicaments and uses of the present invention. CDRs are underlined for mAb7 and mAb15. The mAB7 is also known as RN888 or PF-6801591. mAb7 (aka RN888) and mAb15 are disclosed in International Patent Publication No. WO2016/092419, the disclosure of which is hereby incorporated by reference in its entirety.

TABLE 2

| | |
|---|---|
| mAb7 (aka RN 888) | QVQLVQSGAEVKKPGASVKVSCKASGYTFTSYWINWVRQAPGQGLE |
| or mAb15 full- | WMGNIYPGSSLTNYNEKFKNRVTMTRDTSTSTVYMELSSLRSEDTAV |
| length heavy chain | YYCARLSTGTFAYWGQGTLVTVSSASTKGPSVFPLAPCSRSTSESTA |
| | ALGCLVKDYFPEPVTVSWNSGALTSGVHTFPAVLQSSGLYSLSSVVT |
| | VPSSSLGTKTYTCNVDHKPSNTKVDKRVESKYGPPCPPCPAPEFLGG |
| | PSVFLFPPKPKDTLMISRTPEVTCVVVDVSQEDPEVQFNVVYVDGVEV |
| | HNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVFS |
| | CSVMHEALHNHYTQKSLSLSLGK (SEQ ID NO: 1) |
| mAb7 or mAb 15 | QVQLVQSGAEVKKPGASVKVSCKASGYTFTSYWINWVRQAPGQGLE |
| full-length heavy | WMGNIYPGSSLTNYNEKFKNRVTMTRDTSTSTVYMELSSLRSEDTAV |
| chain without the C- | YYCARLSTGTFAYWGQGTLVTVSSASTKGPSVFPLAPCSRSTSESTA |
| terminal lysine | ALGCLVKDYFPEPVTVSWNSGALTSGVHTFPAVLQSSGLYSLSSVVT |
| | VPSSSLGTKTYTCNVDHKPSNTKVDKRVESKYGPPCPPCPAPEFLGG |
| | PSVFLFPPKPKDTLMISRTPEVTCVVVDVSQEDPEVQFNWYVDGVEV |
| | HNAKTKPREEQFNSTYRVVSVLTVLHQDWLNGKEYKCKVSNKGLPS |
| | SIEKTISKAKGQPREPQVYTLPPSQEEMTKNQVSLTCLVKGFYPSDIA |
| | VEWESNGQPENNYKTTPPVLDSDGSFFLYSRLTVDKSRWQEGNVFS |
| | CSVMHEALHNHYTQKSLSLSLG (SEQ ID NO: 2) |
| mAb7 full-length | DIVMTQSPDSLAVSLGERATINCKSSQSLWDSGNQKNFLTWYQQKP |
| light chain | GQPPKLLIYWTSYRESGVPDRFSGSGSGTDFTLTISSLQAEDVAVYYC |
| | QNDYFYPHTFGGGTKVEIKRGTVAAPSVFIFPPSDEQLKSGTASVVCL |
| | LNNFYPREAKVQWKVDNALQSGNSQESVTEQDSKDSTYSLSSTLTLS |
| | KADYEKHKVYACEVTHQGLSSPVTKSFNRGEC (SEQ ID NO: 3) |
| mAb7 light chain | QVQLVQSGAEVKKPGASVKVSCKASGYTFTSYWINWVRQAPGQGLE |
| variable region | WMGNIYPGSSLTNYNEKFKNRVTMTRDTSTSTVYMELSSLRSEDTAV |
| | YYCARLSTGTFAYWGQGTLVTVSS (SEQ ID NO: 4) |
| mAB7 and mAB15 | QVQLVQSGAEVKKPGASVKVSCKASGYTFTSYWINWVRQAPGQGLE |
| heavy chain | WMGNIWPGSSLTNYNEKFKNRVTMTRDTSTSTVYMELSSLRSEDTA |
| variable region | VYYCARLLTGTFAYWGQGTLVTVSS (SEQ ID NO: 5) |
| mAb15 light chain | DIVMTQSPDSLAVSLGERATINCKSSQSLWDSGNQKNFLTVVYQQKP |
| variable region | GQPPKLLIYWTSYRESGVPDRFSGSGSGTDFTLTISSLQAEDVAVYYC |
| | QNDYFYPHTFGGGTKVEIK (SEQ ID NO: 6) |
| Nivolumab, | QVQLVESGGGWQPGRSLRLDCKASGITFSNSGMHWVRQAPGKGLE |
| MDX1106, full | WVAVrWYDGSKRYYADSVKGRFTISRDNSKNTLFLQMNSLRAEDTAV |
| length heavy chain | YYCATNDDYWGQGTLVTVSSASTKGPSVFPLAPCSRSTSESTAALGC |
| From | LVDYFPEPVTVSWNSGALTSGVHTFPAVLQSSGLYSLSSVVTVPSSS |
| WO2006/121168 | LGTTYTCNVDHKPSNTKVDRVESYGPPCPPCPAPEFLGGPSVFLFPP |
| | KPKDTLMISRTPEVTCWVDVSQEDPEVQFNWYYDGVEVHNATKPRE |
| | EQFNSTYRVVSVLTVLHQDVVLNGKEYKCKVSNKGLPSSIEKTISKA |
| | GQPREPQVYTLPPSQEEMTKNQVSLTCLVKGFYPSDIAVEWESNGQ |
| | PEKNYKTTPPVLDSDGSFFLYSRLTVDKSRWQEGNVFSCSVMHEALH |
| | NHYTQKSLSLSLGK (SEQ ID NO: 7) |
| Nivolumab, | EIVLTQSPATLSLSPGERATLSCRASQSVSSYLAWYQQPGQAPRLLIY |
| MDX1106, full | DASNRATGIPARFSGSGSGTDFTLTISSLEPEDFAVYYCQQSSNWPR |
| length light chain | TFGQGTKVEIRTVAAPSVFIFPPSDEQLSGTASVVCLLNNFYPREAVQ |
| From | WKVDNALQSGNSQESVTEQDSDSTYSLSSTLTLSKADYEKHKVYACE |
| WO2006/121168 | VTHQGLSSPVT SFNRGEC (SEQ ID NO: 8) |
| Pembrolizumab, | QVQLVQSGVEVKKPGASVKVSCKASGYTFTNYYMYVVVRQAPGQ |
| MK3475, full length | GLEWMGGINPSNGGTNFNEKFKNRVTLTTDSSTTTAYMELKSLQF |
| heavy chain | DDTAVYYCARRDYRFDMGFDYWGQGTTVTVSSASTKGPSVFPLA |
| From | PCSRSTSESTAALGCLVKDYFPEPVTVSWNSGALTSGVHTFPAVL |
| WO2009114335 | QSSGLYSLSSVVTVPSSSLGTKTYTCNVDHKPSNTKVDKRVESKY |
| | GPPCPPCPAPEFLGGPSVFLFPPKPKDTLMISRTPEVTCVVVDVSQ |
| | EDPEVQFNWYVDGVEVHNAKTKPREEQFNSTYRVVSVLTVLHQD |
| | WLNGKEYKCKVSNKGLPSSIEKTISKAKGQPREPQVYTLPPSQEE |
| | MTKNQVSLTCLVKGFYPSDIAVEWESNGQPENNYKTTPPVLDSDG |
| | SFFLYSRLTVDKSRWQEGNVFSCSVMHEALHNHYTQKSLSLSLGK |
| | (SEQ ID NO: 9) |
| Pembrolizumab, | EIVLTQSPATLSLSPGERATLSCRASKGVSTSGYSYLHVVYQQKPG |
| MK3475, full length | QAPRLLIYLASYLESGVPARFSGSGSGTDFTLTISSLEPEDFAVYYC |
| light chain | QHSRDLPLTFGGGTKVEIKRTVAAPSVFIFPPSDEQLKSGTASVVC |
| From | LLNNFYPREAKVQWKVDNALQSGNSQESVTEQDSKDSTYSLSSTL |
| WO2009114335 | TLSKADYEKHKVYACEVTHQGLSSPVT KSFNRGEC (SEQ ID NO: |
| | 10) |
| AMP224, without | LFTVTVPKELYIIEHGSNVTLECNFDTGSHVNLGAITASLQKVENDTSP |
| signal sequence | HRERATLLEEQLPLGKASFHIPQVQVRDEGQYQCIIIYGVA |
| From | WDYKYLTLKVKASYRKINTHILKVPETDEVELTCQATGYPLAEVSWPN |
| WO2010027827 | VSVPANTSHSRTPEGLYQVTSVLRLKPPPGRNFSCVFWNTHVRELTL |
| and | ASIDLQSQMEPRTHPTWEPKSCDKTHTCPPCPAPELLGGPSVFLFPP |
| WO2011066342 | KPKDTLMISRTPEVTCWVDVSHEDPEVKFNWYVDGVEVHNAKTKPR |
| | EEQYNSTYRWSVLTVLHQDWLNGKEYKCKVSNKALPAPIEKTISKAK |
| | GQPREPQVYTLPPSRDELTKNQV SLTCLVKGFY PSDIAVEWES |
| | NGQPENNYKT TPPVLDSDGS |
| | FFLYSKLTVDKSRWQQGNVFSCSVMHEALHNHYTQKSLSLSPGK |
| | (SEQ ID NO: 11) |
| YW243.55.S70 | EVQLVESGGGLVQPGGSLRLSCAASGFTFSDSWIHVVVRQAPGKGLE |
| heavy chain | WVAWISPYGGSTYYADSVKGRFTISADTSKNTAYLQMNSLRAEDTAV |
| From | YYCARRHWPGGFDYWGQGTLVTVSA (SEQ ID NO: 12) |
| WO2010077634 | |
| YW243.55.S70 light | DIQMTQSPSSLSASVGDRVTITCRASQDVSTAVAWYQQKPGKAPKLLI |
| chain | YSASFLYSGVPSRFSGSGSGTDFTLTISSLQPEDFATYYCQQYLYH |
| From | PATFGQGTKVEIKR (SEQ ID NO: 13) |
| WO2010077634 | |
| avelumab heavy | EVQLLESGGGLVQPGGSLRLSCAASGFTFSSYIMWVRQAPGKGLEW |
| chain variable | VSSIYPSGGITFYADKGRFTISRDNSKNTLYLQMNSLRAEDTAVYYCA |
| region | RIKLGTVTTVDYWGQ GTLVTVSS (SEQ ID NO: 14) |
| From WO13079174 | |
| avelumab light | QSALTQPASVSGSPGQSITISCTGTSSDVGGYNYVSWYQQHPGKAP |
| chain variable | KLMIYDVSNRPSGVSNRFSGSKSGNTASLTISGLQAEDEADYYCSSYT |
| region | SSSTRVFGTGTKVTVL (SEQ ID NO: 15) |
| From WO13079174 | |

The term “PD-1 binding antagonist” as used herein refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less non-dysfunctional. In some embodiments, the PD-1 binding antagonist is an anti-PD-1 antibody. In a specific aspect, a PD-1 binding antagonist is nivolumab. In another specific aspect, a PD-1 binding antagonist is pembrolizumab. In another specific aspect, a PD-1 binding antagonist is pidilizumab.

The term “PD-L1 binding antagonist” as used herein refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as render a dysfunctional T-cell less non-dysfunctional. In some embodiments, a PD-L1 binding antagonist is an anti-PD-L1 antibody. In a specific aspect, an anti-PD-L1 antibody is avelumab. In another specific aspect, an anti-PD-L1 antibody is atezolizumab. In another specific aspect, an anti-PD-L1 antibody is durvalumab. In another specific aspect, an anti-PD-L1 antibody is BMS-936559 (MDX-1105).

As used herein, an anti-human PD-L1 antibody refers to an antibody that specifically binds to mature human PD-L1. A mature human PD-L1 molecule consists of amino acids 19-290 of the following sequence (SEQ ID NO: 16): MRIFAVFIFMTYWHLLNAFTVTVPKDLYVVEYGSNMTIECKFPVEKQLDLAALIVYWEM EDKNIIQFVHGEEDLKVQHSSYRQRARLLKDQLSLGNAALQITDVKLQDAGVYRCMISY GGADYKRITVKVNAPYNKINQRILVVDPVTSEHELTCQAEGYPKAEVIVVTSSDHQVLSG KTTTTNSKREEKLFNVTSTLRINTTTNElFYCTFRRLDPEENHTAELVIPELPLAHPPNER THLVILGAILLCLGVALTFIFRLRKGRMMDVKKCGIQDTNSKKQSDTHLEET (SEQ ID NO: 16).

Table 3 below provides the sequences of the anti-PD-L1 antibody avelumab for use in the treatment methods, medicaments and uses of the present invention. Avelumab is disclosed as A09-246-2, in International Patent Publication No. WO2013/079174, the disclosure of which is hereby incorporated by reference in its entirety.

TABLE 3
ANTI-HUMAN PD-L1 MONOCLONAL ANTIBODY AVELUMAB
SEQUENCES

| | |
|---|---|
| Heavy chain | SYIMM (SEQ ID NO: 17) |
| CDR1 (CDRH1) | |
| Heavy chain | SIYPSGGITFY (SEQ ID NO: 18) |
| CDR2 (CDRH2) | |
| Heavy chain | IKLGTVTTVDY (SEQ ID NO: 19) |
| CDR3 (CDRH3) | |
| Light chain CDR1 | TGTSSDVGGYNYVS (SEQ ID NO: 20) |
| (CDRL1) | |
| Light chain CDR2 | DVSNRPS (SEQ ID NO: 21) |
| (CDRL2) | |
| Light chain CDR3 | SSYTSSSTRV (SEQ ID NO:22) |
| (CDRL3) | |
| Heavy chain | EVQLLESGGGLVQPGGSLRLSCAASGFTFSSYIMMWVRQAP |
| variable region | GKGLEWVSSIYPSGGITFYADKGRFTISRDNSKNTLYLQMNSL |
| (VR) | RAEDTAVYYCARIKLGTVTTVDYWGQGTLVTVSS (SEQ ID |
| | NO: 14) |
| Light chain VR | QSALTQPASVSGSPGQSITISCTGTSSDVGGYNYVSWYQQHP |
| | GKAPKLMIYDVSNRPSGVSNRFSGSKSGNTASLTISGLQAED |
| | EADYYCSSYTSSSTRVFGTGTKVTVL (SEQ ID NO: 15) |
| Heavy chain | EVQLLESGGGLVQPGGSLRLSCAASGFTFSSYIMMVVVRQAP |
| | GKGLEWVSSIYPSGGITFYADTVKGRFTISRDNSKNTLYLQMN |
| | SLRAEDTAVYYCARIKLGTVTTVDYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPSN |
| | TKVDKKVEPKSCDKTHTCPPCPAPELLGGPSVFLFPPKPKDT |
| | LMISRTPEVTCVVVDVSHEDPEVKFNWYVDGVEVHNAKTKPR |
| | EEQYNSTYRVVSVLTVLHQDWLNGKEYKCKVSNKALPAPIEK |
| | TISKAKGQPREPQVYTLPPSRDELTKNQVSLTCLVKGFYPSDI |
| | AVEWESNGQPENNYKTTPPVLDSDGSFFLYSKLTVDKSRWQ |
| | QGNVFSCSVMHEALHNHYTQKSLSLSPGK (SEQ ID NO: 23) |
| Light chain | QSALTQPASVSGSPGQSITISCTGTSSDVGGYNYVSWYQQHP |
| | GKAPKLMIYDVSNRPSGVSNRFSGSKSGNTASLTISGLQAED |
| | EADYYCSSYTSSSTRVFGTGTKVTVLGQPKANPTVTLFPPSS |
| | EELQANKATLVCLISDFYPGAVTVAWKADGSPVKAGVETTKP |
| | SKQSNNKYAASSYLSLTPEQWKSHRSYSCQVTHEGSTVEKT |
| | VAPTECS (SEQ ID NO: 24) |

The term “PD-L2 binding antagonists” as used herein refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less non-dysfunctional. In some embodiments, a PD-L2 binding antagonist is a PD-L2 immunoadhesin.

A “MEK inhibitor” or a MEKi is a molecule that inhibits the function of mitogen-activated protein kinase kinase 1 (MEK1) or mitogen-activated protein kinase kinase 2 (MEK2) to phosphorylate the extracellular signal-regulated kinases ERK1 and ERK2. In some embodiments, a MEK inhibitor is a small molecule, which is an organic compound that has molecular weight less than 900 Daltons. In some embodiments, the MEK inhibitor is a polypeptide with molecular weight more than 900 Daltons. In some embodiments, the MEK inhibitor is an antibody. Embodiments of a MEK inhibitor include but are not limited to trametinib (aka GSK1120212), cobimetinib (aka Cotellic®, GDC-0973, XL518), refametinib (aka RDEA119, BAY869766), selumetinib (aka AZD6244, ARRY-142886), binimetinib (aka MEK162, ARRY-438162), PD0325901, PD184352 (CI-1040), PD098059, U0126, CH4987655 (aka RO4987655), CH5126755 (aka RO5126766), and GDC623, and any pharmaceutically acceptable salt thereof, as described in C. J. Caunt et al, Nature Reviews Cancer, Volume 15, October 2015, pages 577-592), the disclosure of which is herein incorporated by reference in its entirety.

In one embodiment, the MEK inhibitor is binimetinib, which is 6-(4-bromo-2-fluorophenylamino)-7-fluoro-3-methyl-3H-benzoimidazole-5-carboxylic acid (2-hydroxyethoxy)-amide, and has the following structure.

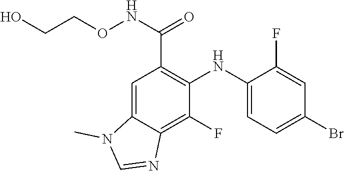 [SMILES: Cn1cnc2c(F)c(Nc3ccc(Br)cc3F)c(C(=O)NOCCO)cc21]

Binimetinib is also known as ARRY-162 and MEK162. Methods of preparing binimetinib and its pharmaceutically acceptable salts, are described in PCT publication No. WO 03/077914, in Example 18 (compound 29111), the disclosure of which is herein incorporated by reference in its entirety. In one embodiment, the MEK inhibitor is binimetinib or a pharmaceutically acceptable salt thereof. In one embodiment, the MEK inhibitor is binimetinib as the free base. In one embodiment, the MEK inhibitor is a pharmaceutically acceptable salt of binimetinib. In one embodiment, the MEK inhibitor is crystallized binimetinib. Crystallized binimetinib and methods of preparing crystallized binimetinib are described in PCT publication No. WO 2014/063024, the disclosure of which is herein incorporated by reference in its entirety.

A “PARP inhibitor” or a “PARPi” is a molecule that inhibits the function of poly(adenosine diphosphate [ADP]-ribose) polymerase (PARP) to repair the single stranded breaks (SSBs) of the DNA. In some embodiments, a PARP inhibitor is a small molecule, which is an organic compound that has molecular weight less than 900 Daltons. In some embodiments, the PARP inhibitor is a polypeptide with molecular weight more than 900 Daltons. In some embodiments, the PARP inhibitor is an antibody. In some embodiments, the PARP inhibitor is selected from the group consisting of olaparib, niraparib, BGB-290, talazoparib, or any pharmaceutically acceptable salt of olaparib, niraparib, BGB-290 or talazoparib thereof. In an embodiment, the PARP inhibitor is talazoparib, or a pharmaceutically acceptable salt thereof and preferably a tosylate salt thereof. In an embodiment, the PARP inhibitor is talazoparib tosylate.

Talazoparib is a potent, orally available PARP inhibitor, which is cytotoxic to human cancer cell lines harboring gene mutations that compromise deoxyribonucleic acid (DNA) repair, an effect referred to as synthetic lethality, and by trapping PARP protein on DNA thereby preventing DNA repair, replication, and transcription. The compound, talazoparib, which is “(8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one” and “(8S,9R)-5-fluoro-8-(4-fluorophenyl)-9-(1-methyl-1H-1,2,4-triazol-5-yl)-2,7,8,9-tetrahydro-3H-pyrido[4,3,2-de]phthalazin-3-one” (also referred to as “PF-06944076”, “MDV3800”, and “BMN673”) is a PARP inhibitor, having the structure,

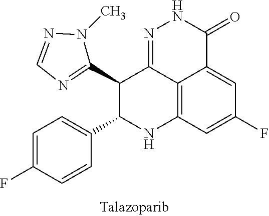 [SMILES: Cn1ncnc1[C@H]1c2n[nH]c(=O)c3cc(F)cc(c23)N[C@@H]1c1ccc(F)cc1]

Talazoparib, and pharmaceutically acceptable salts thereof, including the tosylate salt, are disclosed in International Publication Nos. WO 2010/017055 and WO 2012/054698. Additional methods of preparing talazoparib, and pharmaceutically acceptable salts thereof, including the tosylate salt, are described in International Publication Nos. WO 2011/097602, WO 2015/069851, and WO 2016/019125. Additional methods of treating cancer using talazoparib, and pharmaceutically acceptable salts thereof, including the tosylate salt, are disclosed in International Publication Nos. WO 2011/097334 and WO 2017/075091.

Talazoparib, as a single agent, has demonstrated efficacy, as well as an acceptable toxicity profile in patients with multiple types of solid tumors with DNA repair pathway abnormalities.

“DNA damage response defect positive”, or “DDR defect positive”, as used herein, refers to a condition when an individual or the cancer tissue in the individual is identified as having either germline or somatic genetic alternations in at least one of the DDR genes, as determined by genetic analysis. As used herein, a DDR gene refers to any of those genes that were included in Table 3 of the supplemental material in Pearl et al., Nature Reviews Cancer 15, 166-180 (2015), the disclosure of which is hereby incorporated by reference in its entirety. Exemplary DDR genes include, without limitation, those as described in the below Table 4. Preferred DDR genes include, without limitation, BRCA1, BRCA2, ATM, ATR and FANC. Exemplary genetic analysis includes, without limitation, DNA sequencing, the FoundationOne genetic profiling assay (Frampton et al, Nature Biotechnology, Vol 31, No. 11, 1023-1030, 2013).

TABLE 4
Exemplary DDR genes

| Gene(s) | Description |
|---|---|
| MUTYH (MYH), | Base excision repair (BER) |
| PARP1 (ADPRT), PARP2 (ADPRTL2), | Poly(ADP-ribose) |
| PARP3 (ADPRTL3) | polymerase (PARP) |
| | enzymes that bind to DNA |
| MSH2, MSH6, MLH1, PMS2, | Mismatch excision repair |
| | (MMR) |
| RPA1, ERCC2 (XPD), ERCC4 (XPF) | Nucleotide excision repair |
| | (NER) |
| RAD51, RAD51B, RAD51D, XRCC2, | Homologous |
| XRCC3, RAD52, RAD54L, BRCA1, | recombination |
| RAD50, MRE11A, NBN (NBS1), | |
| FANCA, FANCC, BRCA2 (FANCD1), | Fanconi anemia |
| FANCD2, FANCE, FANCF, FANCG | |
| (XRCC9), FANCI (KIAA1794), FANCL, | |
| FANCM, PALB2 (FANCN), RAD51C | |
| (FANCO), | |
| NUDT1 (MTH1), | Modulation of nucleotide |
| | pools |
| POLD1, POLE, | DNA polymerases |
| | (catalytic subunits) |
| ATM | Genes defective in diseases |
| | associated with sensitivity to |
| | DNA damaging agents |
| ATR, CHEK1, CHEK2, TP53BP1 | Other conserved DNA |
| (53BP1) | damage response genes |

“Loss of heterozygosity score” or “LOH score” as used here in, refers to the percentage of genomic LOH in the tumor tissues of an individual. Percentage genomic LOH, and the calculation thereof are described in Swisher et al (The Lancet Oncology, 18(1):75-87, January 2017), the disclosure of which is incorporated herein by reference in its entirety. Exemplary genetic analysis includes, without limitation, DNA sequencing, and Foundation Medicine's NGS-based T5 assay.

“Homologous recombination deficiency score” or “HRD score” as used here in, refers to the unweighted numeric sum of loss of heterozygosity (“LOH”), telomeric allelic imbalance (“TAI”) and large-scale state transitions (“LST”) in the tumor tissues of an individual. HRD score, together with LOH, and LOH score, and the calculation thereof are described in Timms et al, Breast Cancer Res 2014 Dec. 5; 16(6):475, Telli et al Clin Cancer Res; 22(15); 3764-73.2016, the disclosures of which are incorporated herein by reference in their entireties. Exemplary genetic analysis includes, without limitation, DNA sequencing, Myriad's HRD or HRD Plus assay (Mirza et al N Engl J Med 2016 Dec. 1; 375(22):2154-2164, 2016).

The terms “KRAS-associated cancer”, “HRAS-associated cancer”, and “NRAS-associated cancer” as used herein, refer to cancers associated with or having a dysregulation of a KRAS, HRAS or NRAS gene, respectively, a KRAS, HRAS or NRAS protein, respectively, or expression or activity, or level of the same.

The phrase “dysregulation of a KRAS, HRAS or NRAS gene, a KRAS, HRAS or NRAS kinase, or the expression or activity or level of the same” refers to a genetic mutation or a genetic alteration (e.g., a germline mutation, a somatic mutation, or a recombinant mutation) of a wildtype KRAS, HRAS, or NRAS gene (e.g., a point mutation (e.g., a substitution, insertion, and/or deletion of one or more nucleotides in a wildtype KRAS, HRAS, or NRAS gene); a chromosomal mutation of a wildtype KRAS, HRAS or NRAS gene (e.g., an inversion of a wildtype KRAS, HRAS or NRAS gene; a wildtype KRAS, HRAS, or NRAS gene translocation that results in the expression of a KRAS, HRAS, or NRAS fusion protein, respectively; a deletion in a KRAS, HRAS or NRAS gene that results in the absence of a KRAS, HRAS, or NRAS gene or gene fragment, respectively; a KRAS, HRAS, or NRAS gene duplication (also called amplification) that results in increased levels of a KRAS, HRAS or NRAS protein, respectively; a copy number variation of a KRAS, HRAS, or NRAS gene that results in the expression of a KRAS, HRAS, or NRAS protein having a deletion of at least one amino acid as compared to the wildtype KRAS, HRAS, or NRAS protein; and an expanding trinucleotide repeat of a KRAS, HRAS or NRAS gene); an alternatively spliced version of a KRAS, HRAS, or NRAS mRNA; or an autocrine activity resulting from the overexpression of a KRAS, HRAS or NRAS gene. Other types of genetic mutations or genetic modifications that can cause dysregulation of KRAS, HRAS, or NRAS are described in, e.g., Clancy, S., Genetic mutation, Nature Education 1(1): 187, (2008), the disclosure of which is herein incorporated by reference in its entirety. For example, a dysregulation of a KRAS, HRAS or NRAS gene, a KRAS, HRAS or NRAS protein, or expression or activity, or level of the same, can be a genetic mutation in a wildtype KRAS, HRAS or NRAS gene, respectively, that results in the production of a KRAS, HRAS, or NRAS protein, respectively, that is constitutively active or has increased activity (e.g., overactive) as compared to a protein encoded by a wildtype KRAS, HRAS or NRAS gene, respectively. As another example, a dysregulation of a KRAS, HRAS or NRAS gene, a KRAS, HRAS or NRAS protein, or expression or activity, or level of the same, can be the result of a gene or chromosome translocation which results in the expression of a fusion protein that contains a first portion of KRAS, HRAS, or NRAS, respectively, that includes a functional kinase domain, and a second portion of a partner protein (i.e., that is not KRAS, HRAS, or NRAS, respectively). In some examples, dysregulation of a KRAS, HRAS or NRAS gene, a KRAS, HRAS or NRAS protein, or expression or activity, can be a result of a gene translocation of one KRAS, HRAS or NRAS gene, respectively, with another KRAS, HRAS, or NRAS RAF gene, respectively.

“KRAS mutant cancer”, “HRAS mutant cancer” or “NRAS mutant cancer”, as used herein, refers to a cancer wherein the cancer tissue in the individual is identified as having at least one germline or somatic genetic mutations in the KRAS, HRAS and NRAS gene respectively, as determined by genetic analysis, and wherein such mutation results in overactive mutated KRAS, HRAS and NRAS protein, or such mutation is in the form of increased copies of the wildtype or mutated KRAS, HRAS and NRAS gene on the corresponding chromosome, respectively. As used herein, the mutated KRAS, HRAS and NRAS protein is considered over active if the binding constant Kᵢ of its binding to GTP is at least about 10%, about 20%, about 30%, about 50%, about 100%, about 150%, about 200%, about 300%, about 500%, 10 times, 50 times, or 100 times higher than the binding constant Ki of the corresponding wild type KRAS, HRAS, NRAS protein binding to GTP, respectively. In some embodiments, the genetic mutation of the KRAS gene, HRAS gene or NRAS gene is at codon 12, 13, 59, 61, 117 or 146. In some embodiments, the mutation is a point mutation at codon 12, 13 or 61. In some embodiments, the genetic mutation is a missense mutation at codon 12, 13 or 61. In some embodiments, the genetic mutation of the KRAS gene is selected from the group consisting of G12C, G12R, G12S, G12A, G12D, G12V, G13C, G13R, G13S, G13A, G13D, Q61K, Q61L, Q61R and Q61H in non-small cell lung cancer. In some embodiments, the genetic mutation of the KRAS gene is selected from the group consisting of G12D, G12V, G12R, G12A, G13D, Q61H and Q61L in pancreatic cancer. In some embodiments, the mutation of the KRAS gene, HRAS gene and NRAS gene is in the form of increased copies of the KRAS, HRAS and NRAS gene on the corresponding chromosome locus. Exemplary genetic analysis includes, without limitation, DNA sequencing, and genetic analysis assays approved by a regulatory agency. The term “RAS mutant cancer”, as used herein, refers to cancer that is KRAS mutant cancer, HRAS mutant cancer or HRAS mutant cancer.

“Genetic mutation”, or “genetic alteration”, as used here in, refer to a germline, somatic or recombinant mutation of a wild type gene, including point mutation, chromosomal mutation and copy number variation, wherein point mutation includes substitution, insertion, and deletion of a nucleotide in the gene, chromosomal mutation includes inversion, deletion, duplication, and translocation of the relevant region of the chromosome, and copy number variation includes increased copies of genes on the relevant locus or expanding trinucleotide repeat, as described in Clancy, S., Genetic mutation, Nature Education 1(1):187, (2008), the disclosure of which is herein incorporated by reference in its entirety.

The term “tumor proportion score” or “TPS” as used herein refers to the percentage of viable tumor cells showing partial or complete membrane staining in an immunohistochemistry test of a sample. “Tumor proportion score of PD-L1 expression” as used here in refers to the percentage of viable tumor cells showing partial or complete membrane staining in a PD-L1 expression immunohistochemistry test of a sample. Exemplary samples include, without limitation, a biological sample, a tissue sample, a formalin-fixed paraffin-embedded (FFPE) human tissue sample and a formalin-fixed paraffin-embedded (FFPE) human tumor tissue sample. Exemplary PD-L1 expression immunohistochemistry tests include, without limitation, the PD-L1 IHC 22C3 PharmDx (FDA approved, Daco), Ventana PD-L1 SP263 assay, and the tests described in international patent application PCT/EP2017/073712.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. In one embodiment, the cancer is renal cell carcinoma. In one embodiment, the cancer is pancreatic ductal adenocarcinoma (PDAC).

The term “combination therapy” as used herein refers to any dosing regimen of the therapeutically active agents, (i.e., combination partners), a combination of a MEK inhibitor and a PD-1 axis binding antagonist, or a combination of a MEK inhibitor and a PARP inhibitor, or a combination of a MEK inhibitor and a PD-1 axis binding antagonist and a PARP inhibitor, encompassed in single or multiple compositions, wherein the therapeutically active agents are administered together or separately (each or in any combinations thereof) in a manner prescribed by a medical care taker or according to a regulatory agency as defined herein.

In one embodiment, a combination therapy comprises a combination of a MEK inhibitor and a PD-1 axis binding antagonist and a PARP inhibitor.

In one embodiment, a combination therapy comprises a combination of a MEK inhibitor and a PD-1 axis binding antagonist.

In one embodiment, a combination therapy comprises a combination of a MEK inhibitor and a PARP inhibitor.

In one embodiment, a combination therapy comprises a combination of a MEK inhibitor, which is binimetinib or a pharmaceutically acceptable salt thereof, a PD-1 axis binding antagonist which is avelumab, and a PARP inhibitor which is talazoparib tosylate.

In one embodiment, a combination therapy comprises a combination of a MEK inhibitor which is binimetinib or a pharmaceutically acceptable salt thereof and a PARP inhibitor which is talazoparib or a pharmaceutically acceptable salt thereof.

In one embodiment, a combination therapy comprises a combination of a MEK inhibitor which is binimetinib or a pharmaceutically acceptable salt thereof, and a PD-1 axis binding antagonist which is avelumab.

A “patient” to be treated according to this invention includes any warm-blooded animal, such as, but not limited to human, monkey or other lower-order primate, horse, dog, rabbit, guinea pig, or mouse. For example, the patient is human. Those skilled in the medical art are readily able to identify individuals who are afflicted with cancer and who are in need of treatment.

In some embodiments, the subject has been identified or diagnosed as having a cancer with dysregulation of a KRAS, HRAS or NRAS gene, a KRAS, HRAS or NRAS protein, or expression or activity, or level of the same (e.g., a KRAS, HRAS or NRAS-associated cancer) (e.g., as determined using a regulatory agency-approved, e.g., FDA-approved, assay or kit). In some embodiments, the subject has a tumor that is positive for dysregulation of a KRAS, HRAS or NRAS gene, a KRAS, HRAS or NRAS protein, or expression or activity, or level of the same (e.g., as determined using a regulatory agency-approved assay or kit). The subject can be a subject with a tumor(s) that is positive for dysregulation of a KRAS, HRAS or NRAS gene, a KRAS, HRAS or NRAS protein, or expression or activity, or level of the same (e.g., identified as positive using a regulatory agency-approved, e.g., FDA-approved, assay or kit). The subject can be a subject whose tumors have dysregulation of a KRAS, HRAS or NRAS gene, a KRAS, HRAS or NRAS protein, or expression or activity, or a level of the same (e.g., where the tumor is identified as such using a regulatory agency-approved, e.g., FDA-approved, kit or assay). In some embodiments, the subject is suspected of having a KRAS, HRAS or NRAS-associated cancer. In some embodiments, the subject has a clinical record indicating that the subject has a tumor that has dysregulation of a KRAS, HRAS or NRAS gene, a KRAS, HRAS or NRAS protein, or expression or activity, or level of the same (and optionally the clinical record indicates that the subject should be treated with any of the combinations provided herein). In some embodiments, the subject is a pediatric patient. In one embodiment, the subject has a KRAS-mutant cancer. In one embodiment, the subject has KRAS mutant non-small cell lung cancer. In one embodiment, the subject has KRAS mutant pancreatic ductal adenocarcinoma. In one embodiment, the subject has KRAS mutant colorectal cancer. In one embodiment, the subject has KRAS mutant gastric cancer.

The term “pediatric patient” as used herein refers to a patient under the age of 16 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994.

The terms “treatment regimen”, “dosing protocol” and “dosing regimen” are used interchangeably to refer to the dose and timing of administration of each therapeutic agent in a combination of the invention.

“Ameliorating” means a lessening or improvement of one or more symptoms as compared to not administering a treatment. “Ameliorating” also includes shortening or reduction in duration of a symptom.

As used herein, an “effective dosage” or “effective amount” or “therapeutically effective amount” of a drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of a drug, compound, or pharmaceutical composition is an amount sufficient to accomplish prophylactic or therapeutic treatment either directly or indirectly. As is understood in the clinical context, an effective dosage of a drug, compound, or pharmaceutical composition may be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved. In reference to the treatment of cancer, an effective amount refers to that amount which has the effect of (1) reducing the size of the tumor, (2) inhibiting (that is, slowing to some extent, preferably stopping) tumor metastasis emergence, (3) inhibiting to some extent (that is, slowing to some extent, preferably stopping) tumor growth or tumor invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in patients with these specific tumors, which may be measured as prolongation of the time before disease progression

The term “Q2W” as used herein means once every two weeks.

The term “BID” as used herein means twice a day.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “objective response” or “OR” refers to a measurable response, including complete response (CR) or partial response (PR). An “objective response rate” (ORR) refers to the proportion of patients with tumor size reduction of a predefined amount and for a minimum time period. Generally, ORR refers to the sum of complete response (CR) rate and partial response (PR) rate.

“Complete response” or “CR” as used herein means the disappearance of all signs of cancer (e.g., disappearance of all target lesions) in response to treatment. This does not always mean the cancer has been cured.

As used herein, “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

“Sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

As used herein, “progression-free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

In some embodiments, the anti-cancer effects of the described methods of treating cancer, including, but not limited to “objective response”, “complete response”, “partial response”, “progressive disease”, “stable disease”, “progression free survival”, “duration of response”, as used herein, are as defined and assessed by the investigators using RECIST v1.1 (Eisenhauer et al, Eur J of Cancer 2009; 45(2):228-47) in patients with locally advanced or metastatic solid tumors other than metastatic castration-resistant prostate cancer (CRPC), and RECIST v1.1 and PCWG3 (Scher et al, J Clin Oncol 2016 Apr. 20; 34(12):1402-18) in patients with metastatic CRPC. The disclosures of Eisenhauer et al, Eur J of Cancer 2009; 45(2):228-47 and Scher et al, J Clin Oncol 2016 Apr. 20; 34(12):1402-18 are herein incorporated by references in their entireties.

In some embodiments, the anti-cancer effect of the treatment, including, but not limited to “immune-related objective response” (irOR), “immune-related complete response” (irCR), “immune-related partial response” (irCR), “immune-related progressive disease” (irPD), “immune-related stable disease” (irSD), “immune-related progression free survival” (irPFS), “immune-related duration of response” (irDR), as used herein, are as defined and assessed by Immune-related response criteria (irRECIST, Nishino et. al. J Immunother Cancer 2014; 2:17) for patients with locally advanced or metastatic solid tumors other than patients with metastatic CRPC. The disclosure of Nishino et. al. J Immunother Cancer 2014; 2:17 is herein incorporated by reference in its entirety.

As used herein, “overall survival” (OS) refers to the percentage of individuals in a group who are likely to be alive after a particular duration of time.

By “extending survival” is meant increasing overall or progression-free survival in a treated patient relative to an untreated patient (i.e. relative to a patient not treated with the medicament).

As used herein, “drug related toxicity”, “infusion related reactions” and “immune related adverse events” (“irAE”), and the severity or grades thereof are as exemplified and defined in the National Cancer Institute's Common Terminology Criteria for Adverse Events v 4.0 (NCI CTCAE v 4.0).

As used herein, “in combination with”, or “in conjunction with”, refers to the administration of two, three or more compounds, components or targeted agents concurrently, sequentially or intermittently as separate dosage, or alternatively, as a fixed dose combination of all or part of, for example, all two of, all three of, any two of the three of, the underlying compounds, components or targeted agents. It is understood that any compounds, components, and targeted agents within a fixed dose combination have the same dose regimen and route of delivery.

A “low-dose amount”, as used herein, refers to an amount or dose of a substance, agent, compound, or composition, that is lower than the amount or dose typically used in a clinical setting.

The term “advanced”, as used herein, as it relates to solid tumors, includes locally advanced (non-metastatic) disease and metastatic disease. Locally advanced solid tumors, which may or may not be treated with curative intent, and metastatic disease, which cannot be treated with curative intent are included within the scope of “advanced solid tumors, as used in the present invention. Those skilled in the art will be able to recognize and diagnose advanced solid tumors in a patient.

“Duration of Response” for purposes of the present invention means the time from documentation of tumor model growth inhibition due to drug treatment to the time of acquisition of a restored growth rate similar to pretreatment growth rate.

The term “additive” is used to mean that the result of the combination of two compounds, components or targeted agents is no greater than the sum of each compound, component or targeted agent individually. The term “additive” means that there is no improvement in the disease condition or disorder being treated over the use of each compound, component or targeted agent individually.

The term “synergy” or “synergistic” is used to mean that the result of the combination of two or more compounds, components or targeted agents is greater than the sum of each agent together. The term “synergy” or “synergistic” means that there is an improvement in the disease condition or disorder being treated, over the use of each compound, component or targeted agent individually. This improvement in the disease condition or disorder being treated is a “synergistic effect”. A “synergistic amount” or “synergistically effective amount” is an amount of the combination of the two compounds, components or targeted agents that results in a synergistic effect, as “synergistic” is defined herein. Determining a synergistic interaction between two or more components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods. Exemplary synergistic effects includes, but are not limited to, enhanced efficacy, decreased dosage at equal or increased level of efficacy, reduced or delayed development of drug resistance, and simultaneous enhancement or equal therapeutic actions and reduction of unwanted actions, over the use of each compound, component or targeted agent individually, as described in Jia Jia et al Nature Reviews, Drug Discovery, Volume 8, February 2009, page 111-128, the disclosure of which is herein incorporated by reference in its entirety.

In some embodiments, “synergistic effect” as used herein refers to combination of two or three components or targeted agents for example, a combination of a MEK inhibitor and a PD-1 axis binding antagonist, a combination of a MEK inhibitor and a PARP inhibitor, or a combination of a MEK inhibitor and a PD-1 axis binding antagonist and a PARP inhibitor, producing an effect, for example, slowing the symptomatic progression of a proliferative disease, particularly cancer, or symptoms thereof, which is greater than the simple addition of the effects of each compound, component or targeted agent administered by itself.

A “chemotherapeutic agent” is a chemical compound useful in the treatment of cancer. Examples of chemotherapeutic agents include alkylating agents such as thiotepa and cyclophosphamide (CYTOXAN®); alkyl sulfonates such as busulfan, improsulfan, and piposulfan; aziridines such as. benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; pemetrexed; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma I I and calicheamicin omegaI I (see, e.g., Nicolaou et ai, Angew. Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-it inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDIS1NE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel, also known as nab-paclitaxel (ABRAXANE™) and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Additional examples of chemotherapeutic agents include anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY 1 1 7018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRFI) agonists such as leuprolide acetate (LUPRON® and ELIGARD®), goserelin acetate, buserelin acetate and tripterelin; anti-androgens such as fiutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RJVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); anti-sense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALL® VECTIN® vaccine, LEUVECTIN® vaccine, and VAXID® vaccine; topoisomerase 1 inhibitor (e.g., LURTOTECAN®); an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH (e.g., ABARELIX®); lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutically acceptable salts, acids or derivatives of any of the above.

A “chemotherapy” as used herein, refers to a chemotherapeutic agent, as defined above, or a combination of two, three or four chemotherapeutic agents, for the treatment of cancer. When a chemotherapy consists more than one chemotherapeutic agents, the chemotherapeutic agents can be administered to the patient on the same day or on different days in the same treatment cycle.

A “platinum-based chemotherapy” as used herein, refers to a chemotherapy wherein at least one chemotherapeutic agent is a coordination complex of platinum. Exemplary platinum-based chemotherapy includes, without limitation, cisplatin, carboplatin, oxaliplatin, nedaplatin, gemcitabine in combination with cisplatin, carboplatin in combination with pemetremed.

A “platinum-based doublet” as used herein, refers to a chemotherapy comprising two and no more than two chemotherapeutic agents and wherein at least one chemotherapeutic agent is a coordination complex of platinum. Exemplary platinum-based doublet includes, without limitation, gemcitabine in combination with cisplatin, carboplatin in combination with pemetrexed.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-Ia, IL-2, IL-3, IL-4, IL-5, IL-6, IL-7, IL-8, IL-9, IL-10, IL-1 1, IL-12, IL-13, IL-15, IL-17A-F, IL-18 to IL-29 (such as IL-23), IL-31, including PROLEUKIN® rIL-2; a tumor-necrosis factor such as TNF-a or TNF-β, TGF-I-3; and other polypeptide factors including leukemia inhibitory factor (“LIF”), ciliary neurotrophic factor (“CNTF”), CNTF-like cytokine (“CLC”), cardiotrophin (“CT”), and kit ligand (“L”).

As used herein, the term “chemokine” refers to soluble factors (e.g., cytokines) that have the ability to selectively induce chemotaxis and activation of leukocytes. They also trigger processes of angiogenesis, inflammation, wound healing, and tumorigenesis. Example chemokines include IL-8, a human homolog of murine keratinocyte chemoattractant (KC).

The phrase “pharmaceutically acceptable” indicates that the substance or composition must be compatible chemically and/or toxicologically, with the other ingredients comprising a formulation, and/or the mammal being treated therewith. Some embodiments relate to the pharmaceutically acceptable salts of the compounds described herein. The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002). Methods for making pharmaceutically acceptable salts of compounds described herein are known to one of skill in the art.

The term “solvate” is used herein to describe a molecular complex comprising a compound described herein and one or more pharmaceutically acceptable solvent molecules, for example, water and ethanol.

The compounds described herein may also exist in unsolvated and solvated forms. Accordingly, some embodiments relate to the hydrates and solvates of the compounds described herein.

Compounds described herein containing one or more asymmetric carbon atoms can exist as two or more stereoisomers. Where a compound described herein contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds described herein containing, for example, an imino, keto, or oxime group, or so-called valence tautomerism in compounds which contain an aromatic moiety. A single compound may exhibit more than one type of isomerism.

The compounds of the embodiments described herein include all stereoisomers (e.g., cis and trans isomers) and all optical isomers of compounds described herein (e.g., R and S enantiomers), as well as racemic, diastereomeric and other mixtures of such isomers. While all stereoisomers are encompassed within the scope of our claims, one skilled in the art will recognize that particular stereoisomers may be preferred.

In some embodiments, the compounds described herein can exist in several tautomeric forms, including the enol and imine form, and the keto and enamine form and geometric isomers and mixtures thereof. All such tautomeric forms are included within the scope of the present embodiments. Tautomers exist as mixtures of a tautomeric set in solution. In solid form, usually one tautomer predominates. Even though one tautomer may be described, the present embodiments include all tautomers of the present compounds.

Included within the scope of the present embodiments are all stereoisomers, geometric isomers and tautomeric forms of the compounds described herein, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

The present embodiments also include atropisomers of the compounds described herein. Atropisomers refer to compounds that can be separated into rotationally restricted isomers.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where a compound described herein contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

Exemplary methods and materials are described herein, although methods and materials similar or equivalent to those described herein can also be used in the practice or testing of the invention. The materials, methods, and examples are illustrative only and not intended to be limiting.

### Methods, Uses, and Medicaments

Previous studies by others demonstrated that KRAS and NRAS mutant tumors are highly sensitive to the combination of MEK inhibitor and PARP inhibitor in vitro and for KRAS mutant tumors, in vivo. Sun et al., Sci. Transl. Med. 9, eaal5148 (May, 2017). It has also been shown that PD-L1 expression is correlated with KRAS mutation in lung adenocarcinoma and that the PD-L1 induced apoptosis of CD3+ T cells and mediated immune escape in lung adenocarcinoma cells could be reversed by anti PD-1 antibody pembrolizumab. Chen et al., Cancer Immunol Immunother 66:1175-1187 (April 2017). Furthermore, it has also been shown that combination of a MEK inhibitor and a PD-L1 antibody resulted in synergistic and durable tumor regression even when either agent alone was only modestly effectively. Ebert et al., Immunity 44, 609-621 (March 2016).

In accordance with the present invention, in one embodiment, an amount of a first compound or component, for example, a MEK inhibitor, is used in combination with an amount of a second compound or component, for example, a PD-1 axis binding antagonist and optionally a third compound or component, for example a PARP inhibitor, wherein the amounts together are effective in the treatment of cancer. The amounts, which together are effective, will relieve to some extent one or more of the symptoms of the disorder being treated.

In accordance with the present invention, a therapeutically effective amount of each of the combination partners of a combination therapy of the invention may be administered simultaneously, separately or sequentially and in any order. In one embodiment, a method of treating a proliferative disease, including cancer, may comprise administration of a combination of a MEK inhibitor and a PD-1 axis binding antagonist, or a combination of a MEK inhibitor and a PARP inhibitor, or a combination of a MEK inhibitor and a PD-1 axis binding antagonist and a PARP inhibitor, wherein the individual combination partners are administered simultaneously or sequentially in any order, in jointly therapeutically effective amounts, (for example in synergistically effective amounts), e.g. in daily or intermittently dosages corresponding to the amounts described herein. The individual combination partners of a combination therapy of the invention may be administered separately at different times during the course of therapy or concurrently in divided or single combination forms. In one embodiment, the PARP inhibitor may be administered on a daily basis, either once daily or twice daily, the MEK inhibitor may be administered on a daily basis, either once daily or twice daily, and the PD-1 axis binding antagonist may be administered on a weekly basis. The instant invention is therefore to be understood as embracing all such regimens of simultaneous or alternating treatment and the term “administering” is to be interpreted accordingly.

The term “jointly therapeutically effective amount” as used herein means when the therapeutic agents of a combination described herein are given to the patient simultaneously or separately (e.g., in a chronologically staggered manner, for example a sequence-specific manner) in such time intervals that they show an interaction (e.g., a joint therapeutic effect, for example a synergistic effect). Whether this is the case can, inter alia, be determined by following the blood levels and showing that the combination components are present in the blood of the human to be treated at least during certain time intervals.

In one embodiment, a method of treating a proliferative disease, including cancer, may comprise administration of a MEK inhibitor in free or pharmaceutically acceptable salt form, and administration of a PD-1 axis binding antagonist, simultaneously or sequentially in any order, in jointly therapeutically effective amounts, (for example in synergistically effective amounts), e.g. in daily or corresponding to the amounts described herein. In one embodiment, a method of treating a proliferative disease may comprise administration of a MEK inhibitor in free or pharmaceutically acceptable salt form, administration of a PD-1 axis binding antagonist, and administration of a PARP inhibitor in free or pharmaceutically acceptable salt form, simultaneously or sequentially in any order, in jointly therapeutically effective amounts, (for example in synergistically effective amounts), e.g. in daily or intermittently dosages corresponding to the amounts described herein.

Administration of the compounds or components of the combination of the present invention can be effected by any method that enables delivery of the compounds or components to the site of action. These methods include oral routes, intraduodenal routes, parenteral injection (including intravenous, subcutaneous, intramuscular, intravascular or infusion), topical, and rectal administration.

In one embodiment, provided herein is a method of treating a subject having a proliferative disease comprising administering to said subject a combination therapy as described herein in a quantity which is jointly therapeutically effective against a proliferative disease. In one embodiment, the proliferative disease is cancer. In one embodiment, the cancer is selected from squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer (including renal cell carcinoma), ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer (including pancreatic ductal adenocarcinoma (PDA)), glioblastoma multiforme, cervical cancer, brain cancer, stomach cancer, bladder cancer, hepatoma, breast cancer, colon carcinoma, and head and neck cancer. In one embodiment, the cancer is pancreatic cancer. In one embodiment, the cancer is pancreatic ductal adenocarcinoma (PDA). In one embodiment, the cancer is non-small cell lung cancer. In one embodiment, the cancer is colorectal cancer. In one embodiment, the cancer is gastric cancer. In one embodiment, the cancer is prostate cancer. In one embodiment, the cancer is a RAS mutant cancer. In one embodiment, the cancer is a KRAS mutant cancer. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer. In one embodiment, the cancer is KRAS mutant gastric cancer. In one embodiment, the cancer is a HRAS mutant cancer. In one embodiment, the cancer is a NRAS mutant cancer. In one embodiment, the cancer is DDR defect positive in at least one DDR gene selected from BRCA1, BRCA2, ATM, ATR and FANC. In some embodiments, the subject was previously treated with at least 1 prior line of treatment, e.g., at least 1 treatment with another anticancer treatment, e.g., first- or second-line systemic anticancer therapy (e.g., treatment with one or more cytotoxic agents), resection of a tumor, or radiation therapy. In one embodiment, the prior treatment is platinum-based chemotherapy, docetaxel, a PD-1 axis antagonist, or a combination of chemotherapy with a PD-1 axis antagonist. In one embodiment, the prior treatment is chemotherapy, wherein the chemotherapy is FOLFIRINOX, gemcitabine or gemcitabine in combination with nab-paclitaxel. In one embodiment, the combination therapy comprises a MEK inhibitor, which is binimetinib, a PD-1 axis binding antagonist which is avelumab, and a PARP inhibitor which is talazoparib. In one embodiment, a combination therapy comprises a MEK inhibitor which is binimetinib, and a PD-1 axis binding antagonist which is avelumab.

In one embodiment, provided herein is a method of treating cancer in a patient in need thereof, the method comprising: (a) determining that the cancer in the patient is a KRAS-associated cancer; and (b) administering to the patient a therapeutically effective amount of a combination therapy described herein. In some embodiments, the patient is determined to have a KRAS-associated cancer through the use of a regulatory agency-approved, e.g., FDA-approved test or assay for identifying dysregulation of a KRAS gene, a KRAS kinase, or expression or activity or level of any of the same, in a patient or a biopsy sample from the patient or by performing any of the non-limiting examples of assays described herein. In some embodiments, the test or assay is provided as a kit. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer. In one embodiment, the cancer is KRAS mutant gastric cancer. In one embodiment, the combination therapy comprises a MEK inhibitor, which is binimetinib, a PD-1 axis binding antagonist which is avelumab, and a PARP inhibitor which is talazoparib or a pharmaceutically acceptable salt thereof. In one embodiment, a combination therapy comprises a MEK inhibitor which is binimetinib, and a PD-1 axis binding antagonist which is avelumab.

In one embodiment, the invention provides a method for treating cancer comprising administering to a patient in need thereof therapeutically effective amounts, independently, of a PARP inhibitor, a PD-1 axis binding antagonist, and a MEK inhibitor.

In one embodiment, the invention provides a method for treating cancer comprising administering to a patient in need thereof therapeutically effective amounts, independently, of a PARP inhibitor, a PD-1 axis binding antagonist, and a MEK inhibitor, wherein the PARP inhibitor is talazoparib or a pharmaceutically acceptable salt thereof. In one embodiment, talazoparib or a pharmaceutically acceptable salt thereof is administered orally in the amount of about 0.5 mg QD, about 0.75 mg QD or about 1.0 mg QD. In one embodiment, the PD-1 axis antagonist is avelumab. In one embodiment, avelumab is administered intravenously over 60 minutes in the amount of about 800 mg every 2 weeks (Q2W) or about 10 mg/kg every 2 weeks (Q2W). In one embodiment, the MEK inhibitor is binimetinib or a pharmaceutically acceptable salt thereof. In one embodiment, the MEK inhibitor is binimetinib as the free base. In one embodiment, the MEK inhibitor is crystallized binimetinib. In one embodiment, binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a PARP inhibitor which is talazoparib or a pharmaceutically acceptable salt thereof, (b) a MEK inhibitor, which is binimetinib or a pharmaceutically acceptable salt thereof, and (c) a PD-1 axis binding antagonist which is avelumab. In one embodiment, a method for treating cancer comprises administering to a patient in need thereof a combination therapy comprising therapeutically effective amounts, independently, of (a) a PARP inhibitor which is talazoparib or a pharmaceutically acceptable salt thereof, wherein talazoparib, or a pharmaceutically acceptable salt thereof, is administered orally in the amount of about 0.5 mg QD, about 0.75 mg QD or about 1.0 mg QD, (b) a MEK inhibitor, which is binimetinib or a pharmaceutically acceptable salt thereof, and (c) a PD-1 axis binding antagonist which is avelumab. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a PARP inhibitor which is talazoparib or a pharmaceutically acceptable salt thereof, (b) a MEK inhibitor, which is binimetinib or a pharmaceutically acceptable salt thereof, wherein binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days, and (c) a PD-1 axis binding antagonist which is avelumab. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a PARP inhibitor which is talazoparib or a pharmaceutically acceptable salt thereof, (b) a MEK inhibitor, which is binimetinib or a pharmaceutically acceptable salt thereof, and (c) a PD-1 axis binding antagonist which is avelumab, wherein avelumab is administered intravenously over 60 minutes in the amount of about 800 mg every Q2W or about 10 mg/kg Q2W. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a PARP inhibitor which is talazoparib or a pharmaceutically acceptable salt thereof, wherein talazoparib, or a pharmaceutically acceptable salt thereof, is administered orally in the amount of about 0.5 mg QD, about 0.75 mg QD or about 1.0 mg QD, (b) a MEK inhibitor, which is binimetinib or a pharmaceutically acceptable salt thereof, wherein binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days, and (c) a PD-1 axis binding antagonist which is avelumab, wherein avelumab is administered intravenously over 60 minutes in the amount of about 800 mg every Q2W or about 10 mg/kg Q2W. In one embodiment, the amounts together achieve a synergistic effect in the treatment of cancer.

In one embodiment, the invention provides a method for treating cancer comprising administering to a patient in need thereof therapeutically effective amounts, independently, of a PD-1 axis binding antagonist and a MEK inhibitor.

In one embodiment, the invention provides a method for treating cancer comprising administering to a patient in need thereof therapeutically effective amounts, independently, of an amount of a PD-1 axis binding antagonist, and an amount of a MEK inhibitor. In one embodiment, the PD-1 axis antagonist is avelumab. In one embodiment, avelumab is administered intravenously over 60 minutes in the amount of about 800 mg every 2 weeks (Q2W) or about 10 mg/kg every 2 weeks (Q2W). In one embodiment, the MEK inhibitor is binimetinib or a pharmaceutically acceptable salt thereof. In one embodiment, the MEK inhibitor is crystallized binimetinib. In one embodiment, binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a MEK inhibitor, which is binimetinib or a pharmaceutically acceptable salt thereof, and (b) a PD-1 axis binding antagonist which is avelumab. In one embodiment, a method for treating cancer comprises administering to a patient in need thereof a combination therapy comprising therapeutically effective amounts, independently, of (a) a MEK inhibitor, which is binimetinib or a pharmaceutically acceptable salt thereof, and (b) a PD-1 axis binding antagonist which is avelumab. In one embodiment, the amounts together achieve a synergistic effect in the treatment of cancer.

In one embodiment, a method for treating cancer comprises administering to a patient in need thereof a combination therapy comprising therapeutically effective amounts, independently, of (b) a MEK inhibitor, which is binimetinib or a pharmaceutically acceptable salt thereof, wherein binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days, and (c) a PD-1 axis binding antagonist which is avelumab. In one embodiment, the amounts together achieve a synergistic effect in the treatment of cancer.

In one embodiment, a method for treating cancer comprises administering to a patient in need thereof a combination therapy comprising therapeutically effective amounts, independently, of (a) a MEK inhibitor, which is binimetinib or a pharmaceutically acceptable salt thereof, and (b) a PD-1 axis binding antagonist which is avelumab, wherein avelumab is administered intravenously over 60 minutes in the amount of about 800 mg Q2W or about 10 mg/kg Q2W.

In one embodiment, a method for treating cancer comprises administering to a patient in need thereof a combination therapy comprising therapeutically effective amounts, independently, of (a) a MEK inhibitor, which is binimetinib or a pharmaceutically acceptable salt thereof, wherein binimetinib is orally administered daily in the amount of (i) about 30 mg BID or about 45 mg twice a day (BID), or (ii) orally administered daily in the amount of about 30 mg BID or about 45 mg BID for three weeks followed by one week without administration of binimetinib in at least one treatment cycle of 28 days, and (b) a PD-1 axis binding antagonist which is avelumab, wherein avelumab is administered intravenously over 60 minutes in the amount of about 800 mg Q2W or about 10 mg/kg Q2W. In one embodiment, the amounts together achieve a synergistic effect in the treatment of cancer.

In an embodiment, the invention is related to a method for treating cancer comprising administering to a patient in need thereof an amount of a MEK inhibitor, an amount of a PD-1 axis binding antagonist, and/or an amount of a PARP inhibitor, that is effective in treating cancer. In another embodiment, the invention is related to combination of a MEK inhibitor, a PD-1 axis binding antagonist, and/or a PARP inhibitor, for use in the treatment of cancer. In another embodiment, the invention is related to a method for treating cancer comprising administering to a patient in need thereof an amount of a MEK inhibitor, an amount of a PD-1 axis binding antagonist, and/or an amount of a PARP inhibitor, wherein the amounts together achieve synergistic effects in the treatment of cancer. In another embodiment, the invention is related to a combination of a MEK inhibitor, a PD-1 axis binding antagonist, and/or a PARP inhibitor, for the treatment of cancer, wherein the combination is synergistic. In one embodiment, the method or use of the invention is related to a synergistic combination of targeted therapeutic agents, specifically a MEK inhibitor, in combination with a PD-1 axis binding antagonist, and/or a PARP inhibitor. In one aspect of all the embodiments of this paragraph, the MEK inhibitor is binimetinib or a pharmaceutically acceptable salt thereof, the PARP inhibitor is talazoparib or a pharmaceutically acceptable salt thereof and preferably a tosylate salt thereof, the PD-1 axis binding antagonist is avelumab.

Those skilled in the art will be able to determine, according to known methods, the appropriate amount, dose or dosage of each compound, as used in the combination of the present invention, to administer to a patient, taking into account factors such as age, weight, general health, the compound administered, the route of administration, the nature and advancement of the cancer requiring treatment, and the presence of other medications.

The practice of the method of this invention may be accomplished through various administration or dosing regimens. The compounds of the combination of the present invention can be administered intermittently, concurrently or sequentially. In an embodiment, the compounds of the combination of the present invention can be administered in a concurrent dosing regimen.

Repetition of the administration or dosing regimens may be conducted as necessary to achieve the desired reduction or diminution of cancer cells. A “continuous dosing schedule”, as used herein, is an administration or dosing regimen without dose interruptions, e.g., without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule. In an embodiment, the compounds of the combination of the present invention can be administered concurrently in a continuous dosing schedule.

In one embodiment, the MEK inhibitor is binimetinib or a pharmaceutically acceptable salt thereof. In one embodiment, the MEK inhibitor is crystallized binimetinib. In one embodiment, binimetinib is orally administered. In one embodiment, binimetinib is formulated as a tablet. In one embodiment, a tablet formulation of binimetinib comprises 15 mg of binimetinib or a pharmaceutically acceptable salt thereof. In one embodiment, a tablet formulation of binimetinib comprises 15 mg of crystallized binimetinib. In one embodiment, crystallized binimetinib is orally administered twice daily. In one embodiment, crystallized binimetinib is orally administered twice daily, wherein the second dose of crystallized binimetinib is administered about 12 hours after the first dose of binimetinib. In one embodiment, 30 mg of crystallized binimetinib is orally administered twice daily. In one embodiment, 45 mg of crystallized binimetinib is orally administered twice daily.

In one embodiment, 45 mg of crystallized binimetinib is orally administered twice daily until observation of adverse effects, after which 30 mg of crystallized binimetinib is administered twice daily. In one embodiment, patients who have been dose reduced to 30 mg twice daily may re-escalate to 45 mg twice daily if the adverse effects that resulted in a dose reduction improve to baseline and remain stable for, e.g., up to 14 days, or up to three weeks, or up to 4 weeks, provided there are no other concomitant toxicities related to binimetinib that would prevent drug re-escalation.

In an embodiment, the PARP inhibitor is talazoparib, or a pharmaceutically acceptable salt thereof and preferably a tosylate thereof, and is administered once daily to comprise a complete cycle of 28 days. Repetition of the 28 day cycles is continued during treatment with the combination of the present invention.

In an embodiment, talazoparib, or a pharmaceutically acceptable salt thereof and preferably a tosylate thereof, is administered once daily to comprise a complete cycle of 21 days. Repetition of the 21 day cycles is continued during treatment with the combination of the present invention.

In an embodiment, talazoparib, or a pharmaceutically acceptable salt thereof and preferably a tosylate thereof, is orally administered at a daily dosage of from about 0.1 mg to about 2 mg once a day, preferably from about 0.25 mg to about 1.5 mg once a day, and more preferably from about 0.5 to about 0.01 mg once a day. In an embodiment, talazoparib or a pharmaceutically acceptable salt thereof and preferably a tosylate thereof, is administered at a daily dosage of about 0.5 mg, 0.75 mg or 1.0 mg once daily. Dosage amounts provided herein refer to the dose of the free base form of talazoparib, or are calculated as the free base equivalent of an administered talazoparib salt form. For example, a dosage or amount of talazoparib, or a pharmaceutically acceptable salt thereof, such as 0.5, 0.75 mg or 1.0 mg refers to the free base equivalent. This dosage regimen may be adjusted to provide the optimal therapeutic response. For example, the dose may be proportionally reduced or increased as indicated by the exigencies of the therapeutic situation.

In some embodiments, the PD-1 axis binding antagonist is avelumab and will be administered intravenously at a dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In some embodiment, avelumab is administered as a flat dose of about 80, 150, 160, 200, 240, 250, 300, 320, 350, 400, 450, 480, 500, 550, 560, 600, 640, 650, 700, 720, 750, 800, 850, 880, 900, 950, 960, 1000, 1040, 1050, 1100, 1120, 1150, 1200, 1250, 1280, 1300, 1350, 1360, 1400, 1440, 1500, 1520, 1550 or 1600 mg, preferably 800 mg, 1200 mg or 1600 mg at intervals of about 14 days (±2 days) or about 21 days (±2 days) or about 30 days (±2 days) throughout the course of treatment. In certain embodiments, a subject will be administered an intravenous (IV) infusion of a medicament comprising any of the PD-1 axis binding antagonists described herein. In one embodiment, avelumab is administered in an amount of 10 mg/kg as an intravenous infusion over 60 minutes every two weeks. In one embodiment, the patient is premedicated with acetaminophen and an antihistamine prior to intravenous infusion of avelumab. In one embodiment, the patient is premedicated with acetaminophen and an antihistamine for the first 4 infusions of avelumab and subsequently as needed. In certain embodiment, the subject will be administered a subcutaneous (SC) infusion of a medicament comprising any of the PD-1 axis binding antagonist described herein.

In one embodiment, any of the dosing regimens of a combination therapy as described herein comprising a MEK inhibitor, a PD-1 axis binding antagonist and a PARP inhibitor, a therapeutically effective amount of the PARP inhibitor is taken together with the first therapeutically effective dose of the MEK inhibitor. As used herein, the phrase “taken together with” means that not more than 5 minute, or not more than 10 minutes, or not more than 15 minutes, or not more than 20 minutes, or not more than 25 minutes, or not more than 30 minutes have passed between the administration of PARP inhibitor and MEK inhibitor.

In one embodiment, any of the dosing regimens of a combination therapy as described herein, the second therapeutically effective dose of the MEK inhibitor is administered about 12 hours after the administration of the first dose of the MEK inhibitor. As used herein, the phrase “about 12 hours after the administration of the first dose of the MEK inhibitor” means that the second dose of the MEK inhibitor is administered 10 to 14 hours after the administration of the first dose of the MEK inhibitor.

In one embodiment, of any of the dosing regimens of a combination therapy as described herein, on days when the PD-1 axis binding antagonist is administered, the PD-1 axis binding antagonist is administered at least 30 minutes after the latter of the administration of a therapeutically effective amount of the PARP inhibitor (if the combination therapy comprises a MEK inhibitor, a PD-1 axis binding antagonist and a PARP inhibitor) and the first therapeutically effective dose of the MEK inhibitor wherein the MEK inhibitor is administered twice daily. As used herein, the phrase “at least 30 minutes after” means that the PD-1 axis binding antagonist is administered at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes after the latter of administration of the PARP inhibitor (if part of the combination therapy) and the first dose of the MEK inhibitor.

In one embodiment, of any of the dosing regimens of a combination therapy as described herein, on days when the PD-1 axis binding antagonist is administered, the PD-1 axis binding antagonist is administered at least 30 minutes, before the administration of a therapeutically effective amount of the PARP inhibitor (if the combination therapy comprises a MEK inhibitor, a PD-1 axis binding antagonist and a PARP inhibitor) and the first therapeutically effective dose of the MEK inhibitor. As used herein, the phrase “at least 30 minutes after” means that the PD-1 axis binding antagonist is administered at least 30 minutes, or at least 35 minutes, or at least 40 minutes, or at least 45 minutes, or at least 50 minutes, or at least 55 minutes, or at least 60 minutes, or at least 65 minutes, or at least 70 minutes, or at least 75 minutes, or at least 80 minutes, or at least 85 minutes, or at least 90 minutes before of administration of the PARP inhibitor (if part of the combination therapy) and the first dose of the MEK inhibitor.

In one embodiment, any combination therapy described herein further comprises administration of one or more pre-medications prior to the administration of the PD-1 axis binding antagonist. In one embodiment, the one or more pre-medication(s) is administered no sooner than 1 hour after administration of the PARP inhibitor (if the combination therapy comprises a MEK inhibitor, a PD-1 axis binding antagonist and a PARP inhibitor) and the MEK inhibitor. In one embodiment, the one or more premedication(s) is administered 30-60 minutes prior to the administration of the PD-1 axis binding antagonist. In one embodiment, the one or more premedication(s) is administered 30 minutes prior administration of the PD-1 axis binding antagonist. In one embodiment, the one or more pre-medications is selected from one or more of a H₁ antagonist (e.g., antihistamines such as diphenhydramine) and acetaminophen.

In one embodiment, provided herein is a method (e.g., in vitro method) of selecting a treatment for a patient identified or diagnosed as having a KRAS-associated cancer. Some embodiments can further include administering the selected treatment to the patient identified or diagnosed as having a KRAS-associated cancer. For example, the selected treatment can include administration of a therapeutically effective amount of a combination therapy. Some embodiments can further include a step of performing an assay on a sample obtained from the patient to determine whether the patient has a dysregulation of a KRAS gene, a KRAS kinase, or expression or activity or level of any of the same, and identifying and diagnosing a patient determined to have a dysregulation of a KRAS gene, a KRAS kinase, or expression or activity or level of any of the same, as having a KRAS-associated cancer. In some embodiments, the patient has been identified or diagnosed as having a KRAS-associated cancer through the use of a regulatory agency-approved, e.g., FDA-approved, kit for identifying dysregulation of a KRAS gene, a KRAS kinase, or expression or activity or level of any of the same, in a patient or a biopsy sample from the patient. In some embodiments, the KRAS-associated cancer is a cancer described herein or known in the art. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer or a KRAS mutant gastric cancer. In some embodiments, the assay is an in vitro assay, for example, an assay that utilizes the next generation sequencing, immunohistochemistry, or break apart FISH analysis. In some embodiments, the assay is a regulatory agency-approved, e.g., FDA-approved, kit.

The term “regulatory agency” is a country's agency for the approval of the medical use of pharmaceutical agents with the country. For example, a non-limiting example of a regulatory agency is the U.S. Food and Drug Administration (FDA).

Also provided are methods of treating a patient that include performing an assay on a sample obtained from the patient to determine whether the patient has a KRAS-associated cancer (e.g., a cancer having a KRAS mutation), and administering a therapeutically effective amount of a combination therapy to the patient determined to have KRAS-associated cancer (e.g., a cancer having a KRAS kinase mutation). In some embodiments, the KRAS-associated cancer is a cancer described herein or known in the art. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer or a KRAS mutant gastric cancer. In some embodiments, the assay is an in vitro assay, for example, an assay that utilizes the next generation sequencing, immunohistochemistry, or break apart FISH analysis. In some embodiments, the assay is a regulatory agency-approved, e.g., FDA-approved, kit. In some embodiments, the patient was previously treated with at least 1 prior line of treatment, e.g., at least 1 treatment with another anticancer treatment, e.g., first- or second-line systemic anticancer therapy (e.g., treatment with one or more cytotoxic agents), resection of a tumor, or radiation therapy. In one embodiment, the prior treatment is platinum-based chemotherapy, docetaxel, a PD-1 axis antagonist, or a combination of chemotherapy with a PD-1 axis antagonist. In one embodiment, the prior treatment is chemotherapy, wherein the chemotherapy is FOLFIRINOX, gemcitabine or gemcitabine in combination with nab-paclitaxel. In one embodiment, the combination therapy comprises a MEK inhibitor, which is binimetinib, a PD-1 axis binding antagonist which is avelumab, and a PARP inhibitor which is talazoparib. In one embodiment, a combination therapy comprises a MEK inhibitor which is binimetinib, and a PD-1 axis binding antagonist which is avelumab.

In one embodiment, provided herein is a method of treating a subject having a KRAS-associated cancer (e.g., a cancer having a KRAS mutation), said method comprising administering to said subject a therapeutically effective amount of a combination therapy described herein, wherein the subject was treated with at least 1 prior line of treatment prior to treatment with a combination therapy described herein. In one embodiment, the patient has been treated with, e.g., at least 1 treatment with another anticancer treatment, e.g., first- or second-line systemic anticancer therapy (e.g., treatment with one or more cytotoxic agents), resection of a tumor, or radiation therapy. In one embodiment, the prior treatment is platinum-based chemotherapy, docetaxel, a PD-1 axis antagonist, or a combination of chemotherapy with a PD-1 axis antagonist. In one embodiment, the prior treatment is chemotherapy, wherein the chemotherapy is FOLFIRINOX, gemcitabine or gemcitabine in combination with nab-paclitaxel. In some embodiments, the KRAS-associated cancer is a cancer described herein or known in the art. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer or a KRAS mutant gastric cancer. In one embodiment, the combination therapy comprises a MEK inhibitor, which is binimetinib, a PD-1 axis binding antagonist which is avelumab, and a PARP inhibitor which is talazoparib. In one embodiment, a combination therapy comprises a MEK inhibitor which is binimetinib, and a PD-1 axis binding antagonist which is avelumab.

An improvement in a cancer or cancer-related disease can be characterized as a complete or partial response. “Complete response” or “CR” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions.

Treatment may be assessed by inhibition of disease progression, inhibition of tumor growth, reduction of primary tumor, relief of tumor-related symptoms, inhibition of tumor secreted factors (including expression levels of checkpoint proteins as identified herein), delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), improved Time to tumor response (TTR), increased duration of response (DR), increased Progression Free Survival (PFS), increased Overall Survival (OS), Objective Response Rate (ORR), among others. OS as used herein means the time from treatment onset until death from any cause. TTP as used herein means the time from treatment onset until tumor progression; TTP does not comprise deaths. As used herein, TTR is defined for patients with confirmed objective response (CR or PR) as the time from the date of randomization or date of first dose of study treatment to the first documentation of objective tumor response. As used herein, DR means the time from documentation of tumor response to disease progression. As used herein, PFS means the time from treatment onset until tumor progression or death. As used herein, ORR means the proportion of patients with tumor size reduction of a predefined amount and for a minimum time period, where response duration usually is measured from the time of initial response until documented tumor progression. In the extreme, complete inhibition, is referred to herein as prevention or chemoprevention.

Thus, provided herein are methods for achieving one or more clinical endpoints associated with treating a cancer with a combination therapy described herein. In one embodiment, a patient described herein can show a positive tumor response, such as inhibition of tumor growth or a reduction in tumor size after treatment with a combination described herein. In certain embodiments, a patient described herein can achieve a Response Evaluation Criteria in Solid Tumors (for example, RECIST 1.1) of complete response, partial response or stable disease after administration of an effective amount a combination therapy described herein. In certain embodiments, a patient described herein can show increased survival without tumor progression. In some embodiments, a patient described herein can show inhibition of disease progression, inhibition of tumor growth, reduction of primary tumor, relief of tumor-related symptoms, inhibition of tumor secreted factors (including tumor secreted hormones, such as those that contribute to carcinoid syndrome), delayed appearance of primary or secondary tumors, slowed development of primary or secondary tumors, decreased occurrence of primary or secondary tumors, slowed or decreased severity of secondary effects of disease, arrested tumor growth and regression of tumors, decreased Time to Tumor Response (TTR), increased Duration of Response (DR), increased Progression Free Survival (PFS), increased Time To Progression (TTP), and/or increased Overall Survival (OS), among others. In one embodiment, the combination therapy comprises a MEK inhibitor, which is binimetinib, a PD-1 axis binding antagonist which is avelumab, and a PARP inhibitor which is talazoparib. In one embodiment, a combination therapy comprises a MEK inhibitor which is binimetinib, and a PD-1 axis binding antagonist which is avelumab.

In another embodiment, methods are provided for decreasing Time to Tumor Response (TTR), increasing Duration of Response (DR), increasing Progression Free Survival (PFS) of a patient having a cancer described herein, comprising administering an effective amount of a combination therapy as described herein. In one embodiment, a method is provided for decreasing Time to Tumor Response (TTR) of a patient having a cancer described herein, comprising administering an effective amount of a combination therapy as described herein. In one embodiment, is a method for increasing Progression Free Survival (PFS) of a patient a cancer described herein, comprising administering an effective amount of a combination therapy as described herein. In one embodiment, is a method for increasing Progression Free Survival (PFS) of a patient having a cancer described herein, comprising administering an effective amount of a combination therapy as described herein. In one embodiment, the cancer is In one embodiment, the cancer is a KRAS mutant cancer. In one embodiment, the cancer is KRAS mutant non-small cell lung cancer. In one embodiment, the cancer is KRAS mutant pancreatic ductal adenocarcinoma. In one embodiment, the cancer is KRAS mutant colorectal cancer. In one embodiment, the cancer is KRAS mutant gastric cancer. In one embodiment, the combination therapy comprises a MEK inhibitor, which is binimetinib, a PD-1 axis binding antagonist which is avelumab, and a PARP inhibitor which is talazoparib. In one embodiment, a combination therapy comprises a MEK inhibitor which is binimetinib, and a PD-1 axis binding antagonist which is avelumab.

In some embodiments of any of the methods or uses described herein, an assay used to determine whether the patient has a KRAS-associated cancer using a sample from a patient can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof. Assays can utilize other detection methods known in the art for detecting dysregulation of a KRAS gene, a KRAS kinase, or expression or activity or levels of any of the same (see, e.g., the references cited herein). In some embodiments, the sample is a biological sample or a biopsy sample (e.g., a paraffin-embedded biopsy sample) from the patient. In some embodiments, the patient is a patient suspected of having a KRAS-associated cancer, a patient having one or more symptoms of a KRAS-associated cancer, and/or a patient that has an increased risk of developing a KRAS-associated cancer).

In one embodiment, the methods of treating cancer according to the invention also include surgery or radiotherapy. Non-limiting examples of surgery include, e.g., open surgery or minimally invasive surgery. Surgery can include, e.g., removing an entire tumor, debulking of a tumor, or removing a tumor that is causing pain or pressure in the subject. Methods for performing open surgery and minimally invasive surgery on a subject having a cancer are known in the art. Non-limiting examples of radiation therapy include external radiation beam therapy (e.g., external beam therapy using kilovoltage X-rays or megavoltage X-rays) or internal radiation therapy. Internal radiation therapy (also called brachytherapy) can include the use of, e.g., low-dose internal radiation therapy or high-dose internal radiation therapy. Low-dose internal radiation therapy includes, e.g., inserting small radioactive pellets (also called seeds) into or proximal to a cancer tissue in the subject. High-dose internal radiation therapy includes, e.g., inserting a thin tube (e.g., a catheter) or an implant into or proximal to a cancer tissue in the subject, and delivering a high dose of radiation to the thin tube or implant using a radiation machine. Methods for performing radiation therapy on a subject having a cancer are known in the art.

It may be shown by established test models that a combination therapy described herein results in the beneficial effects described herein before. The person skilled in the art is fully enabled to select a relevant test model to prove such beneficial effects. The pharmacological activity of a combination therapy described herein may, for example, be demonstrated in a clinical study or in a test procedure, for example as described below.

Suitable clinical studies are, for example, open label, dose escalation studies in patients with a proliferative disease. Such studies may demonstrate in particular the synergism of the therapeutic agents of a combination therapy described herein. The beneficial effects on proliferative diseases may be determined directly through the results of these studies. Such studies may, in particular, be suitable for comparing the effects of a monotherapy using any one of the MEK inhibitor, the PD-1 axis binding antagonist or the PARP inhibitor versus the effects of a triple combination therapy comprising the MEK inhibitor, the PD-1 axis binding antagonist and the PARP inhibitor, or for comparing the effects of dual therapy using any two of the MEK inhibitor, the PD-1 axis binding antagonist and the PARP inhibitor versus the effects of a monotherapy using any one of the MEK inhibitor, the PD-1 axis binding antagonist or the PARP inhibitor.

In one embodiment wherein the combination therapy is a triplet therapy comprising a MEK inhibitor, PD-1 axis binding antagonist, and a PARP inhibitor, the dose of the MEK inhibitor is escalated until the Maximum Tolerated Dosage is reached, and the PD-1 axis binding antagonist and the PARP inhibitor are each administered as a fixed dose. Alternatively, the MEK inhibitor and the PARP inhibitor may be administered as a fixed dose and the dose of the PD-1 axis binding antagonist may be escalated until the Maximum Tolerated Dosage is reached. Alternatively, the dose of the MEK inhibitor and the PD-1 axis binding antagonist may each be administered as a fixed dose and the dose of the PARP inhibitor may be escalated until the Maximum Tolerated Dosage is reached.

In one embodiment wherein the combination therapy is a doublet therapy comprising a MEK inhibitor and a PD-1 axis binding antagonist, the dose of the MEK inhibitor is escalated until the Maximum Tolerated Dosage is reached, and the PD-1 axis binding antagonist is administered as a fixed dose. Alternatively, the MEK inhibitor may be administered as a fixed dose and the dose of the PD-1 axis binding antagonist may be escalated until the Maximum Tolerated Dosage is reached.

The efficacy of the treatment may be determined in such studies, e.g., after 6, 12, 18 or 24 weeks by evaluation of symptom scores, e.g., every 6 weeks.

The compounds of the method or combination of the present invention may be formulated prior to administration. The formulation will preferably be adapted to the particular mode of administration. These compounds may be formulated with pharmaceutically acceptable carriers as known in the art and administered in a wide variety of dosage forms as known in the art. In making the pharmaceutical compositions of the present invention, the active ingredient will usually be mixed with a pharmaceutically acceptable carrier, or diluted by a carrier or enclosed within a carrier. Such carriers include, but are not limited to, solid diluents or fillers, excipients, sterile aqueous media and various non-toxic organic solvents. Dosage unit forms or pharmaceutical compositions include tablets, capsules, such as gelatin capsules, pills, powders, granules, aqueous and nonaqueous oral solutions and suspensions, lozenges, troches, hard candies, sprays, creams, salves, suppositories, jellies, gels, pastes, lotions, ointments, injectable solutions, elixirs, syrups, and parenteral solutions packaged in containers adapted for subdivision into individual doses.

Parenteral formulations include pharmaceutically acceptable aqueous or nonaqueous solutions, dispersion, suspensions, emulsions, and sterile powders for the preparation thereof. Examples of carriers include water, ethanol, polyols (propylene glycol, polyethylene glycol), vegetable oils, and injectable organic esters such as ethyl oleate. Fluidity can be maintained by the use of a coating such as lecithin, a surfactant, or maintaining appropriate particle size. Exemplary parenteral administration forms include solutions or suspensions of the compounds of the invention in sterile aqueous solutions, for example, aqueous propylene glycol or dextrose solutions. Such dosage forms can be suitably buffered, if desired.

Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols.

When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

In one embodiment, the MEK inhibitor is formulated for oral administration. In one embodiment, the MEK inhibitor is formulated as a tablet or capsule. In one embodiment, the MEK inhibitor is formulated as a tablet. In one embodiment, the tablet is a coated tablet. In one embodiment, the MEK inhibitor is binimetinib or a pharmaceutically acceptable salt thereof. In one embodiment, the MEK inhibitor is binimetinib as the fee base. In one embodiment, the MEK inhibitor is a pharmaceutically acceptable salt of binimetinib. In one embodiment, the MEK inhibitor is crystallized binimetinib. Methods of preparing oral formulations of binimetinib are described in PCT publication No. WO 2014/063024. In one embodiment, a tablet formulation of binimetinib comprises 15 mg of binimetinib. In one embodiment, a tablet formulation of binimetinib comprises 15 mg of crystallized binimetinib. In one embodiment, a tablet formulation of binimetinib comprises 45 mg of binimetinib. In one embodiment, a tablet formulation of binimetinib comprises 45 mg of crystallized binimetinib.

The invention also relates to a kit comprising the therapeutic agents of the combination of the present invention and written instructions for administration of the therapeutic agents. In one embodiment, the written instructions elaborate and qualify the modes of administration of the therapeutic agents, for example, for simultaneous or sequential administration of the therapeutic agents of the present invention. In one embodiment, the written instructions elaborate and qualify the modes of administration of the therapeutic agents, for example, by specifying the days of administration for each of the therapeutic agents during a 28 day cycle.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

### EXAMPLE

### Example 1: Clinical Study of the Combination of Binimetinib and Avelumab, with or without Talazoparib, for the Treatment of Cancer

This is a Phase 1/2, open label, multi-center, study of binimetinib in combination with avelumab with or without talazoparib in adult patients with locally advanced or metastatic KRAS mutant NSCLC, and pancreatic ductal adenocarcinoma (PDAC) and other KRAS mutant solid tumors. As used in this Example, the term “talazoparib” refers to talazoparib or any pharmaceutically acceptable salt thereof, including but not limited to talazoparib tosylate.

Phase 1b of Binimetinib in Combination with Avelumab:

The safety and preliminary anti-tumor activity of the binimetinib plus avelumab combination will be evaluated in this phase 1/2 portion of the study in patients with KRAS mutant NSCLC and PDAC.

Initially, 2 cohorts of patients with KRAS mutant NSCLC and PDAC will be enrolled and treated with binimetinib at 45 mg BID or 30 mg BID administered orally in combination with avelumab administered at the fixed dose of 800 mg IV Q2W in 28 day cycles and evaluated for DLT during Cycle 1, as shown in Table 5.

TABLE 5
Avelumab and Binimetinib dose levels

| Dose | Avelumab dose IV | Binimetinib dose oral |
|---|---|---|
| level | (mg Q2W) | (mg BID) |
| D0 | 800 | 45 |
| D1 | 800 | 30 |

If DLTs are observed the binimetinib dose may be reduced or alternative dosing schedules for binimetinib (3 weeks on and 1 week off) may be explored should the emerging safety data suggest that continuous BID dosing is not tolerable.

Phase 1b Binimetinib in Combination with Avelumab and Talazoparib:

A phase 1 dose-finding portion will identify the recommended phase 2 dose (RP2D) of the binimetinib and talazoparib in the triplet combination. Patients with locally advanced or metastatic KRAS mutant NSCLC and PDAC may be treated with 2 different doses (30 or 45 mg) of binimetinib administered orally twice a day (BID) and 3 different doses of talazoparib (0.5 mg, 0.75 mg, or 1.0 mg) administered orally every day (QD), and a fixed dose of avelumab (800 mg Q2W), as shown in Table 6, in a 28 day treatment cycle and will be evaluated for dose limiting toxicities (DLTs).

TABLE 6
Avelumab, Binimetinib and Talazoparib dose levels

| | Avelumab | Binimetinib | Talazoparib |
|---|---|---|---|
| Dose | dose IV | dose oral | dose oral |
| level | (mg Q2W) | (mg BID) | (mg QD) |
| D0 | 800 | 30 | 0.5 |
| D1 | 800 | 30 | 0.75 |
| D2 | 800 | 45 | 0.5 |
| D3 | 800 | 45 | 0.75 |
| D4 | 800 | 30 | 1.0 |
| D5 | 800 | 45 | 1.0 |

The DLT evaluation period will be 28 days (i.e., Cycle 1) and the modified toxicity probability interval (mTPI) method will be used to define the RP2D for the combination. Alternative dosing schedules for binimetinib (3 weeks on and 1 week off) may be also explored should the emerging safety data suggest that continuous BID dosing is not tolerable. In addition, the combination of talazoparib plus binimetinib may be evaluated, including using the relevant dosing regimens in Table 6, if the triplet combination is not tolerable.

Phase 2 Design

Once the Phase 1 b is completed and the R2PD for the doublet (binimetinib in combination with avelumab) and the triplet (binimetinib in combination with avelumab and talazoparib) have been determined, the Phase 2 portion will be initiated to evaluate the safety and anti-tumor activity of the RP2D for each combination. Patients for the KRAS mutant NSCLC and mPDAC cohorts will be randomized in a 1:1 ratio to the doublet and the triplet. In addition patients with other KRAS mutant advanced solid tumors will be enrolled to receive the triplet treatment.

Assessment of Tumor Response, Safety and Biomarkers

Overall response rate (ORR) of binimetinib in combination with avelumab with or without talazoparib, will be assessed per Response Evaluation Criteria in Solid Tumors, version 1.1 (RECIST v1.1) in the patients in the study.

Safety, Overall Survival (OS), and other antitumor activity data such as time to tumor response (TTR), duration of response (DR), and progression-free survival (PFS) will be assessed using RECIST v1.1.

The correlation of anti-tumor activity of the combinations with PD-L1 expression, DDR gene alterations, PI3K/mTOR pathway activation markers such as PIK3CA mutations and PTEN deletions will be evaluated.

Potential predictive and/or pharmacodynamic biomarkers in peripheral blood and tumor tissue that may be relevant to the mechanism of action of or resistance to binimetinib and avelumab with or without talazoparib, including but not limited to, biomarkers related to the immune response will also be evaluated.

1. A method for treating cancer comprising administering to a patient in need thereof an amount of a PARP inhibitor, an amount of a PD-1 axis binding antagonist, and an amount of a MEK inhibitor, wherein the amounts together are effective in treating cancer.

2. The method of claim 1, wherein the cancer in the patient is a RAS mutant cancer.

3. The method of claim 2, wherein the cancer in the patient is a KRAS mutant cancer, a HRAS mutant cancer, or a NRAS mutant cancer.

4. (canceled)

5. The method of claim 1, wherein the cancer is pancreatic cancer, a non-small lung cancer, colorectal cancer, or gastric cancer.

6-8. (canceled)

9. The method of claim 1, wherein the PD-1 axis antagonist is an anti PD-1 antibody selected from the group consisting of nivolumab, pembrolizumab, and RN888.

10. The method of claim 1, wherein the PD-1 axis antagonist is an anti PD-L1 antibody selected from the group consisting of avelumab, durvalumab and atezolizumab.

11. The method of claim 1, wherein the PARP inhibitor is selected from the group consisting of olaparib, niraparib, BGB-290 and talazoparib, or a pharmaceutically acceptable salt thereof.

12. The method of claim 1, wherein the MEK inhibitor is selected from the group consisting of trametinib, cobimetinib, refametinib, selumetinib, binimetinib, PD0325901, PD184352, PD098059, U0126, CH4987655, CH5126755 and GDC623, or a pharmaceutically acceptable salt thereof.

13-22. (canceled)

23. The method of claim 1, wherein the PARP inhibitor is talazoparib or a pharmaceutically acceptable salt thereof and is administered orally in the amount of about 0.5 mg QD, about 0.75 mg QD or about 1.0 mg QD, the PD-1 axis antagonist is avelumab and is administered intravenously in the amount of about 800 mg Q2W or about 10 mg/kg Q2W, and the MEK inhibitor is binimetinib or a pharmaceutically acceptable salt thereof and is administered orally in the amount of (a) about 30 mg BID or about 45 mg BID, or (b) about 30 mg BID or about 45 mg BID for three weeks on and one week off in at least one treatment cycle of 28 days.

24-32. (canceled)

33. A method for treating cancer comprising administering to a patient in need thereof an amount of a PD-1 axis binding antagonist, and an amount of a MEK inhibitor, wherein the PD-1 axis antagonist is avelumab, and the MEK inhibitor is binimetinib or a pharmaceutically acceptable salt thereof, wherein the amounts together are effective in treating cancer.

34. The method of claim 33, wherein avelumab is administered intravenously in the amount of about 800 mg Q2W or about 10 mg/kg Q2W, and binimetinib or a pharmaceutically acceptable salt thereof is administered orally in the amount of (a) about 30 mg BID or about 45 mg BID, or (b) about 30 mg BID or about 45 mg BID for three weeks on and one week off in at least one treatment cycle of 28 days.

35-40. (canceled)

41. A method for treating cancer comprising administering to a patient in need thereof an amount of a PARP inhibitor, and an amount of a MEK inhibitor, wherein the PARP inhibitor is talazoparib or a pharmaceutically acceptable salt thereof, and the MEK inhibitor is binimetinib or a pharmaceutically acceptable salt thereof, wherein the amounts together are effective in treating cancer.

42. The method of claim 41, wherein talazoparib or a pharmaceutically acceptable salt thereof is administered orally in the amount of about 0.5 mg QD, about 0.75 mg QD or about 1.0 mg QD, and binimetinib or a pharmaceutically acceptable salt thereof is administered orally in the amount of (a) about 30 mg BID or about 45 mg BID, or (b) about 30 mg BID or about 45 mg BID for three weeks on and one week off in at least one treatment cycle of 28 days.

43-48. (canceled)

49. The method of claim 1, wherein the cancer has a tumor proportion score for PD-L1 expression of less than about 1%, or equal or over about 1%, 5%, 10%, 25%, 50%, 75% or 80%.

50. The method of claim 1, wherein the cancer has a loss of heterozygosity score of about 5% or more, 10% or more, 14% or more 15% or more, 20% or more, or 25% or more.

51. The method of claim 1, wherein the cancer is DDR defect positive in at least one DDR gene selected from BRCA1, BRCA2, ATM, ATR, CHK2, PALB2, MRE11A, NMB RAD51C, MLH1, FANCA and FANC.

52. The method of claim 1, wherein the patient has a HRD score of about 20 or above, 25 or above, 30 or above, 35 or above, 40 or above, 42 or above, 45 or above, or 50 or above.

53. The method of claim 1, wherein the

54-59. (canceled)

60. The method of claim 1, wherein the cancer is locally advanced or metastatic non-small cell lung cancer, and the patient has received at least one prior line of treatment for the locally advanced or metastatic non-small cell lung cancer, wherein the cancer is KRAS mutant non-small cell lung cancer.

61. (canceled)

62. The method of claim 1, wherein the cancer is metastatic pancreatic cancer, wherein the patient has received at least one prior line of chemotherapy for the cancer.

63-64. (canceled)
